(19) United States
(12) Patent Application Publication
(10) Pub. No.: US 2022/0133841 A1
(43) Pub. Date: May 5, 2022

(54) NEUROLOGICAL DISEASE TREATMENT WITH ZILUCOPLAN

(71) Applicant: Ra Pharmaceuticals, Inc., Cambridge, MA (US)

(72) Inventors: Petra Duda, Charlestown, MA (US); Ramin Farzaneh-Far, Brookline, MA (US); Zhong Ma, Bedford, MA (US); Nanqun Zhu, Lexington, MA (US); Evan Thackaberry, Groton, MA (US); Alonso Ricardo, Winchester, MA (US)

(21) Appl. No.: 17/287,581

(22) PCT Filed: Oct. 22, 2019
(86) PCT No.: PCT/US2019/057316

§ 371 Date: Apr. 22, 2021

Related U.S. Application Data
(60) Provisional application No. 62/748,659, filed on Oct. 22, 2018.
(60) Provisional application No. 62/777,524, filed on Dec. 10, 2018.
(60) Provisional application No. 62/815,575, filed on Mar. 8, 2019.
(60) Provisional application No. 62/837,974, filed on Apr. 24, 2019.
(60) Provisional application No. 62/844,388, filed on May 7, 2019.
(60) Provisional application No. 62/899,864, filed on Sep. 13, 2019.

(51) Int. Cl. A61K 38/12; A61P 21/04

(52) U.S. Cl. CPC A61K 38/12; A61P 21/04

(57) ABSTRACT

Embodiments of the present disclosure include methods of treating myasthenia gravis, including generalized myasthenia gravis, by providing C5 complement inhibitors. Included are devices and kits for inhibitor administration and methods of evaluating complement inhibitor treatment efficacy.

### CROSS REFERENCE TO RELATED APPLICATIONS

This application claims priority to U.S. Provisional Application No. 62/748,659 filed on Oct. 22, 2018 entitled NEUROLOGICAL DISEASE TREATMENT WITH COMPLEMENT INHIBITORS. U.S. Provisional Application No. 62/777,524 filed on Dec. 10, 2018 entitled NEUROLOGICAL DISEASE TREATMENT WITH COMPLEMENT INHIBITORS, U.S. Provisional Application No. 62/815,575 filed on Mar. 8, 2019 entitled MODULATORS OF COMPLEMENT ACTIVITY, U.S. Provisional Application No. 62/837,974 filed on Apr. 24, 2019 entitled MODULATORS OF COMPLEMENT ACTIVITY, U.S. Provisional Application No. 62/844,388 filed on May 7, 2019 entitled NEUROLOGICAL DISEASE TREATMENT WITH COMPLEMENT INHIBITORS, and U.S. Provisional Application No. 62/899,864 filed on Sep. 13, 2019 entitled NEUROLOGICAL DISEASE TREATMENT WITH COMPLEMENT INHIBITORS, the contents of each of which are herein incorporated by reference in their entirety.

### SEQUENCE LISTING

The instant application contains a Sequence Listing which has been submitted electronically in ASCII format and is hereby incorporated by reference in its entirety. Said ASCII copy, created on Oct. 22, 2019, is named 2011_1035PCT_SL.txt and is 1,286 bytes in size.

### BACKGROUND

The vertebrate immune response is comprised of adaptive and innate immune components. While the adaptive immune response is selective for particular pathogens and is slow to respond, components of the innate immune response recognize a broad range of pathogens and respond rapidly upon infection. One such component of the innate immune response is the complement system.

The complement system includes about 20 circulating complement component proteins, synthesized primarily by the liver. Components of this particular immune response were first termed “complement” due to the observation that they complemented the antibody response in the destruction of bacteria. These proteins remain in an inactive form prior to activation in response to infection. Activation occurs by way of a pathway of proteolytic cleavage initiated by pathogen recognition and leading to pathogen destruction. Three such pathways are known in the complement system and are referred to as the classical pathway, the lectin pathway, and the alternative pathway. The classical pathway is activated when an IgG or IgM molecule binds to the surface of a pathogen. The lectin pathway is initiated by the mannan-binding lectin protein recognizing the sugar residues of a bacterial cell wall. The alternative pathway remains active at low levels in the absence of any specific stimuli. While all three pathways differ with regard to initiating events, all three pathways converge with the cleavage of complement component C3. C3 is cleaved into two products termed C3a and C3b. Of these, C3b becomes covalently linked to the pathogen surface while C3a acts as a diffusible signal to promote inflammation and recruit circulating immune cells. Surface-associated C3b forms a complex with other components to initiate a cascade of reactions among the later components of the complement system. Due to the requirement for surface attachment, complement activity remains localized and minimizes destruction to non-target cells.

Pathogen-associated C3b facilitates pathogen destruction in two ways. In one pathway. C3b is recognized directly by phagocytic cells and leads to engulfment of the pathogen. In the second pathway, pathogen-associated C3b initiates the formation of the membrane attack complex (MAC). In the first step. C3b complexes with other complement components to form the C5-convertase complex. Depending on the initial complement activation pathway, the components of this complex may differ. C5-convertase formed as the result of the classical complement pathway comprises C4b and C2a in addition to C3b. When formed by the alternative pathway. C5-convertase comprises two subunits of C3b as well as one Bb component.

Complement component C5 is cleaved by either C5-convertase complex into C5a and C5b. C5a, much like C3a, diffuses into the circulation and promotes inflammation, acting as a chemoattractant for inflammatory cells. C5b remains attached to the cell surface where it triggers the formation of the MAC through interactions with C6, C7, C8 and C9. The MAC is a hydrophilic pore that spans the membrane and promotes the free flow of fluid into and out of the cell, thereby destroying it.

An important component of all immune activity is the ability of the immune system to distinguish between self and non-self cells. Pathology arises when the immune system is unable to make this distinction. In the case of the complement system, vertebrate cells express proteins that protect them from the effects of the complement cascade. This ensures that targets of the complement system are limited to pathogenic cells. Many complement-related disorders and diseases are associated with abnormal destruction of self cells by the complement cascade. Some complement-related disorders and diseases include neurological diseases and disorders, such as myasthenia gravis.

Myasthenia gravis is a rare complement-mediated autoimmune disease characterized by the production of autoantibodies targeting proteins that are critical for the normal transmission of electrical signals from nerves to muscles. The prevalence of MG in the United States is estimated at approximately 60,000 cases. In approximately 15% of patients with MG, symptoms are confined to the ocular muscles. The remaining patients have MG that affects multiple muscle groups throughout the body, which is typically referred to as generalized MG (gMG). Patients with gMG present with muscle weakness that characteristically becomes more severe with repeated use and recovers with rest. Muscle weakness can be localized to specific muscles, but often progresses to more diffuse muscle weakness. Generalized myasthenia gravis symptoms can become life-threatening when muscle weakness involves the diaphragm and intercostal muscles in the chest wall that are responsible for breathing. The most dangerous complication of gMG, known as myasthenic crisis, requires hospitalization, intubation, and mechanical ventilation. Approximately 15% to 20% of patients with gMG will experience a myasthenic crisis within 2 years of diagnosis.

There remains a need for compositions and methods for treating complement-related diseases and disorders, including those affecting the nervous system, such as myasthenia gravis. The present disclosure meets this need by providing related compositions and methods.

### SUMMARY

In some embodiments, the present disclosure provides a method of treating complement-related indications and/or autoimmune indications and/or neurological disorders as disclosed herein, for example myasthenia gravis (MG), the method comprising administering a compound, or a composition comprising a compound, which modulates complement activity to a subject. Such a compound may be an inhibitor that blocks complement activation (a complement inhibitor), for example a C5 inhibitor, for example a C5 inhibitor polypeptide as described herein. The compound may be zilucoplan or active metabolites or variants thereof, as disclosed herein and the indication or disorder may be MG, as further defined herein below. Herein, MG may include or be generalized MG (gMG). The compound (e.g., zilucoplan) administration may include subcutaneous (SC) administration. The compound (e.g., zilucoplan) may be administered at a dose of from about 0.1 mg/kg (mg compound/kg subject body weight) to about 0.6 mg/kg. Compound administration may include self-administration. Compound administration may include use of a prefilled syringe. The syringe may include a 29-gauge needle. Compound administration may include self-administration using a self-administration device. The self-administration device may include a prefilled syringe. The prefilled syringe may include glass. Optionally, the prefilled syringe is a glass syringe. The prefilled syringe may include a maximum fill volume of at least 1 ml. The self-administration device may include a solution of the compound. The solution may be an aqueous solution. The solution may be preservative-free. The self-administration device may include a solution volume of from about 0.15 ml to about 0.81 ml. The subject may be screened prior to administration of the compound (e.g., zilucoplan). The screening may include assessment of Quantitative Myasthenia Gravis (QMG) score. Subject QMG score may be ≥12. The subject may be prohibited from receiving MG therapy for at least 10 hours prior to QMG score assessment. The MG therapy may be acetylcholinesterase inhibitor therapy. The screening may require that ≥4 QMG test items achieve a score of ≥2. The subject may be between 18 and 85 years old. Screening may include selecting subjects previously diagnosed with gMG. The gMG diagnosis may be made according to Myasthenia Gravis Foundation of America (MGFA) criteria. Screening may include assessment of acetylcholinesterase receptor (AChR) autoantibody levels. Screening may include confirming no change in corticosteroid dose received by the subject for at least 30 days prior to screening. Screening may include confirming no change in subject immunosuppressive therapy for at least 30 days prior to screening. Screening may include a serum pregnancy test and/or a urine pregnancy test. Compound administration may include daily administration. The subject may simultaneously receive standard of care gMG therapy. The standard of care gMG therapy may include one or more of pyridostigmine treatment, corticosteroid treatment, and immunosuppressive drug treatment. The subject may be evaluated or monitored for an MG characteristic, wherein the MG characteristic includes one or more of QMG score, Myasthenia Gravis-Activities of Daily Living (MG-ADL) score, MG-QOL15r score, and MG Composite score. Subject evaluation or monitoring may include assessing change in the MG characteristic during or after subject treatment with a compound as described herein (e.g.: zilucoplan). The MG characteristic may include QMG score reduction. Treated subject QMG score may be reduced by at least 3 points. Treated subject QMG score may be reduced at or before 12 weeks of treatment. Treated subject QMG score may be monitored over the course of the treatment. The subject may receive cholinesterase inhibitor treatment over the course of the treatment. Cholinesterase inhibitor treatment may be withheld for at least 10 hours prior to assessment of treated subject QMG score. Change in MG characteristic may include change in MG Composite score of at least 3 points from a baseline MG Composite score. Change in MG Composite score from baseline MG Composite score may occur at or before 12 weeks of the treatment. Change in MG characteristic may include change in MG-ADL score of at least 2 points from a baseline MG-ADL score. Change in MG-ADL score from baseline MG-ADL score may occur at or before 12 weeks of the treatment. The compound may be in a solution, wherein the solution comprises phosphate-buffered saline (PBS). The solution may include from about 4 mg/ml to about 200 mg/ml of the compound (e.g.: zilucoplan). The solution may include about 40 mg/ml of the compound (e.g., zilucoplan). Subject plasma levels of the compound may reach maximum concentration (Cₘₐₓ) on the first day of treatment. At least 90% hemolysis inhibition may be achieved in subject serum, wherein, optionally, hemolysis inhibition is measured by a sheep red blood cell (sRBC) hemolysis assay. The compound (e.g., zilucoplan) may be administered at a daily dose of from about 0.1 mg/kg to about 0.3 mg/kg. Zilucoplan may be administered at a dose of 0.3 mg/kg. Subject QMG score and/or MG-ADL score may be reduced. QMG score may be reduced by ≥3 points by 8 weeks of treatment. MG-ADL score may be reduced by ≥2 points by 8 weeks of treatment. Risk of need for rescue therapy may be reduced. Administration may be carried out at an MG disease stage that occurs prior to a critical or crisis stage of MG. Administration of the compound (e.g., zilucoplan) may lead to reduced subject symptom expression. The reduced subject symptom expression may exceed reduced subject symptom expression associated with eculizumab administration. Subject neuromuscular junction (NMJ) membrane attack complex (MAC) pore formation may be inhibited. Safety factor at the NMJ may be improved. Zilucoplan may be administered in combination with a therapeutic agent. The therapeutic agent may include an immunosuppressive agent. The immunosuppressive agent may include a compound selected from one or more of azathioprine, cyclosporine, cyclosporine A, mycophenolate mofetil, methotrexate, tacrolimus, cyclophosphamide, and rituximab. The therapeutic agent may include an inhibitor of autoantibody-mediated tissue destruction. The inhibitor of autoantibody-mediated tissue destruction may include a neonatal Fc receptor (FcRN) inhibitor. Administration of the FcRN inhibitor may include intravenous immunoglobulin (IVIG) treatment. Zilucoplan and the therapeutic agent may be administered in overlapping regimens.

In some embodiments, the present disclosure provides a kit that may include a syringe that includes the compound (e.g., zilucoplan) and instructions for use. The syringe may include a self-injection device. The self-injection device may include a BD ULTRASAFE PLUS™ self-administration device. The kit may include an alcohol wipe. The kit may include a wound dressing. The kit may include a disposal container. The compound may be in a solution. The solution may be an aqueous solution. The solution may include phosphate buffered saline. The solution may include from about 4 mg/ml to about 200 mg/ml of the compound (e.g., zilucoplan). The solution may include about 40 mg/ml of the compound (e.g., zilucoplan). The solution may include a preservative.

According to some embodiments, the present disclosure provides a method of evaluating a treatment for a complement-related indications and/or autoimmune indications and/or neurological disorders as disclosed herein, such as for MG. The method may include screening an evaluation candidate for at least one evaluation participation criteria; selecting an evaluation participant; administering the treatment for the indication or disorder (e.g., MG) to the evaluation participant; and assessing at least one efficacy endpoint, wherein, optionally, the treatment involves administration of a compound (e.g., zilucoplan) or composition as disclosed herein. The at least one evaluation participation criteria may include MG diagnosis. The MG diagnosis may include gMG diagnosis. The MG diagnosis may be gMG diagnosis. The gMG diagnosis may be made according to MGFA criteria. The at least one evaluation participation criteria may include QMG score. Evaluation participant selection may require an evaluation candidate QMG score of ≥12. The evaluation candidate may have received at least one alternative MG treatment prior to screening. Evaluation candidate QMG score may be assessed at least 10 hours after receiving the at least one alternative MG treatment. The at least one alternative MG treatment may include acetylcholinesterase inhibitor administration. Evaluation participant selection may require a score of ≥2 for ≥4 QMG test items. The at least one evaluation participation criteria may include evaluation candidate age. Evaluation participant selection may require evaluation candidate age of between 18 and 85 years old. The at least one evaluation participation criteria may include AChR autoantibody level and/or anti-muscle-specific kinase autoantibody level. The at least one evaluation participation criteria may include no change in corticosteroid dose received by evaluation candidate for at least 30 days prior to screening. The at least one evaluation participation criteria may include no change in evaluation candidate immunosuppressive therapy for at least 30 days prior to screening. The at least one evaluation participation criteria may include confirmation that the evaluation candidate is not pregnant. Screening may include a serum pregnancy test and/or a urine pregnancy test. The treatment for MG may be administered over an evaluation period. The evaluation period may be from about 1 day to about 12 weeks. The evaluation period may be about 12 weeks or longer. The evaluation participant may receive standard of care gMG therapy over the evaluation period. The standard of care gMG therapy may include one or more of pyridostigmine treatment, corticosteroid treatment, and immunosuppressive drug treatment. The at least one efficacy endpoint may include treated subject QMG score reduction. The treated subject QMG score reduction may be at least 3 points. The evaluation participant may receive cholinesterase inhibitor treatment during the evaluation period. The cholinesterase inhibitor treatment may be withheld for at least 10 hours prior to assessment of treated subject QMG score. The at least one efficacy endpoint may include a change in baseline score for one or more of MG-ADL score. MG-QOL15r score, and MG Composite score. The at least one efficacy endpoint may include a change in baseline MG Composite score of at least 3 points. The change in baseline MG Composite score may occur at or before 12 weeks of the treatment. The at least one efficacy endpoint may include a change in baseline MG-ADL score of at least 2 points. The change in baseline MG-ADL score may occur at or before 12 weeks of the treatment for MG. Assessing the at least one efficacy endpoint may include a set of assessments. The set of assessments may be carried out in the order of: (1) assessing evaluation participant MG-QOL15r score; (2) assessing evaluation participant MG-ADL score; (3) assessing evaluation participant QMG score; and (4) assessing evaluation participant MG Composite score. The set of assessments may be carried out on one or more occasions after administering the treatment for MG. The one or more occasions after administering the treatment for MG may include 1 week, 2 weeks, 4 weeks, 8 weeks, and/or 12 weeks after administering the treatment for MG.

In some embodiments the present disclosure provides an administration device prepared for treatment of a complement-related indication and/or neurological disorder as disclosed herein, such as MG. The administration device may include a self-injection device comprising a syringe and a needle. The administration device may include a predetermined volume of a pharmaceutical composition. The pharmaceutical composition may include a 40 mg/mL concentration of a compound as disclosed herein (e.g., zilucoplan) in an aqueous solution. The predetermined volume may be modified to facilitate administration of the compound (e.g., zilucoplan) to a subject at a dose of 0.3 mg per kg subject weight. The administration device may include a BD ULTRASAFE PLUS™ self-administration device.

In some embodiments, the present disclosure provides a kit prepared for treatment of MG. The kit may include a set of two or more administration devices described herein and instructions for kit usage. The kit may include an alcohol wipe. The kit may include a wound dressing. The kit may include a disposal container. Kit administration devices may include pharmaceutical compositions of the compound (e.g., zilucoplan) that are preservative free. The kit may be prepared for storage at room temperature.

### BRIEF DESCRIPTION OF THE FIGURES

The foregoing and other objects, features and advantages will be apparent from the following description of particular embodiments of the invention, as illustrated in the accompanying drawings. The drawings are not necessarily to scale; emphasis instead being placed upon illustrating the principles of various disclosed embodiments.

FIG. 1 is a schematic showing the overlap between classical and alternative complement pathways.

FIG. 2 is a schematic showing a myasthenia gravis treatment study design.

FIG. 3 is a graph showing quantitative myasthenia gravis (QMG) score change from baseline during twelve weeks of placebo versus 0.3 mg/kg zilucoplan treatment.

FIG. 4 is a graph showing Myasthenia Gravis-Activities of Daily Living (MG-ADL) score change from baseline during twelve weeks of placebo versus 0.3 mg/kg zilucoplan treatment.

FIG. 5 is a graph showing QMG score change from baseline during twelve weeks of placebo versus 0.1 mg/kg zilucoplan treatment.

FIG. 6 is a graph showing MG-ADL score change from baseline during twelve weeks of placebo versus 0.1 mg/kg zilucoplan treatment.

FIG. 7 is a graph showing MG-ADL score change from baseline during twelve weeks of placebo versus combined average score changes from 0.1 mg/kg and 0.3 mg/kg zilucoplan treatment doses.

FIG. 8 is a graph showing point improvement in QMG score by percent of patients for 0.3 mg/kg zilucoplan treatment versus placebo.

FIG. 9 is a graph showing point improvement in MG-ADL score by percent of patients for 0.3 mg/kg zilucoplan treatment versus placebo.

FIG. 10 is a graph showing percent of patients achieving minimal symptom expression at specified durations of treatment based on MG-ADL analysis with zilucoplan treatments versus eculizumab.

FIG. 11 is a graph showing zilucoplan concentration in samples obtained from patients over the course of zilucoplan or placebo treatment.

FIG. 12 is a graph showing percent hemolysis values obtained by hemolysis assay analysis of samples obtained from patients over the course of zilucoplan or placebo treatment.

FIG. 13 is a graph showing hemolysis values plotted against zilucoplan concentration values, where both sets of values are associated with samples from placebo or zilucoplan treated patients.

FIG. 14 is a graph showing change in QMG score from pretreatment baseline values over the course of 12-week placebo treatment or over the course of 24-week zilucoplan treatment (0.1 mg/kg or 0.3 mg/kg dose). Change in QMG score is also shown for subjects receiving zilucoplan treatment (0.3 mg/kg) from weeks 12-24 after switching from placebo treatment, where the 12-week placebo treatment score is used as the baseline for determining change in score.

FIG. 15 is a graph showing change in MG-ADL score from pretreatment baseline values over the course of 12-week placebo treatment or over the course of 24-week zilucoplan treatment (0.1 mg/kg or 0.3 mg/kg dose). Change in MG-ADL score is also shown for subjects receiving zilucoplan treatment (0.3 mg/kg) from weeks 12-24 after switching from placebo treatment, where the 12-week placebo treatment score is used as the baseline for determining change in score.

FIG. 16 is a graph showing change in MG Composite score from pretreatment baseline values over the course of 12-week placebo treatment or over the course of 24-week zilucoplan treatment (0.1 mg/kg or 0.3 mg/kg dose). Change in MG Composite score is also shown for subjects receiving zilucoplan treatment (0.3 mg/kg) from weeks 12-24 after switching from placebo treatment, where the 12-week placebo treatment score is used as the baseline for determining change in score.

FIG. 17 is a graph showing change in MG-QOL15r score from pretreatment baseline values over the course of 12-week placebo treatment or over the course of 24-week zilucoplan treatment (0.1 mg/kg or 0.3 mg/kg dose). Change in MG-QOL15r score is also shown for subjects receiving zilucoplan treatment (0.3 mg/kg) from weeks 12-24 after switching from placebo treatment, where the 12-week placebo treatment score is used as the baseline for determining change in score.

FIG. 18 is a graph showing percentage of each compound tested moving from upper chamber to lower chamber in an in-vitro permeability assay using a basement membrane model.

### DETAILED DESCRIPTION

The present disclosure relates to neurological disorder treatment by inhibiting complement activity. Complement activity protects the body from foreign pathogens but can lead to self-cell destruction with elevated activity or poor regulation. Myasthenia gravis is a neurological disorder characterized by autoantibody-mediated nervous system destruction. Included herein are methods of treating myasthenia gravis by administering complement inhibitors. Also included are methods for testing new treatments for MG. These and other embodiments of the disclosure are described in detail below.

### I. Compounds and Compositions

In some embodiments, the present disclosure provides compounds and compositions comprising said compounds which function to modulate complement activity. Such compounds and compositions may include inhibitors that block complement activation. As used herein, “complement activity” includes the activation of the complement cascade, the formation of cleavage products from a complement component such as C3 or C5, the assembly of downstream complexes following a cleavage event, or any process or event attendant to, or resulting from, the cleavage of a complement component, e.g., C3 or C5. Complement inhibitors may include C5 inhibitors that block complement activation at the level of complement component C5. C5 inhibitors may bind C5 and prevent its cleavage, by C5 convertase, into the cleavage products C5a and C5b. As used herein, “Complement component C5” or “C5” is defined as a complex which is cleaved by C5 convertase into at least the cleavage products, C5a and C5b. “_{C}5 inhibitors,” as referred to herein, include any compound or composition that inhibits the processing or cleavage of the pre-cleaved complement component C5 complex or the cleavage products of the complement component C5.

It is understood that inhibition of C5 cleavage prevents the assembly and activity of the cytolytic membrane attack complex (MAC) on glycosylphosphatidylinositol (GPI) adherent protein-deficient erythrocytes. In some cases. C5 inhibitors presented herein may also bind C5b, preventing C6 binding and subsequent assembly of the C5b-9 MAC.

C5 inhibitor compounds may include, but are not limited to, any of those presented in Table 1. References listed and information supporting listed clinical study numbers are incorporated herein by reference in their entirety.

TABLE 1
C5 inhibitors

| | | | | Clinical study | |
|---|---|---|---|---|---|
| Compound | Company | Target | Compound type | numbers | References |
| Eculizumab | Alexion | C5 | Monoclonal antibody | NCT01303952; | U.S. Pat. No. |
| (SOLIRIS ®) | Pharmaceuticals, | | directed against C5 | NCT02093533; | 6,355,245 |
| | Inc. | | protein. Inhibits C5 | NCT01567085; | |
| | | | cleavage. | NCT01919346; | |
| | | | | NCT01895127; | |
| | | | | NCT01399593; | |
| | | | | NCT02145182; | |
| | | | | NCT01106027; | |
| | | | | NCT02301624; | |
| | | | | NCT01997229; | |
| | | | | NCT01892345 | |
| ALXN1210 | Alexion | C5 | Antibody | NCT02598583; | US |
| | Pharmaceuticals, | | | NCT02605993; | 2016/0168237 |
| | Inc. | | | NCT02946463; | |
| | | | | NCT03056040; | |
| | | | | NCT02949128 | |
| Tesidolumab/L | Novartis | C5 | Antibody | NCT02878616; | U.S. Pat. No. 8,241,628; |
| FG316 | | | | NCT02763644; | U.S. Pat. No. 8,883,158 |
| | | | | NCT01527500; | |
| | | | | NCT02515942; | |
| | | | | NCT02534909; | |
| | | | | NCT01526889 | |
| ALN-CC5 | Alnylam | C5 | Nucleic acid | NCT02352493 | |
| Zimura | Ophthotech | C5 | Nucleic acid | NCT02397954 | |
| | | | | NCT02686658 | |
| Coversin | Akari | C5 | Protein | NCT02591862 | |
| ALKN1007 | Alexion | C5a | Antibody | NCT02245412; | |
| | | | | NCT02128269 | |
| IFX-1 | InflaRx | C5a | Antibody | NCT02246595; | |
| | | | | NCT02866825; | |
| | | | | NCT03001622 | |
| MUBODINA ® | Adienne Pharma | C5 | Antibody | | U.S. Pat No. 7,999,081 |
| ALXN5500 | Alexion | C5 | Antibody | | |
| | Pharmaceuticals, | | | | |
| | Inc. | | | | |
| ISU305 | ISU ABXIS | C5 | Antibody | | |
| Long-acting | Akari | C5 | Protein | | |
| coversin | | | | | |
| SOBI005 | Swedish Orphan | C5 | Protein | | |
| | Biovitrum Ab | | | | |
| IFX-2, IFX-3 | InflaRx | C5a | Antibody | | |
| NOX-D21 | Noxxon | C5a | Spiegelmer | | |
| rEV576 | Volution | C5 | Antibody | | Penabad et al., |
| | Immunopharmaceuticals | | | | Lupus, 2014 |
| | | | | | 23(12):1324-6 |
| ARC1005 | Novo Nordisk | C5 | Antibody | | |
| SOMAmers | SomaLogic | C5 | Antibody | | |

### Peptide-Based Compounds

In some embodiments, C5 inhibitors of the present disclosure are polypeptides. According to the present disclosure, any amino acid-based molecule (natural or non-natural) may be termed a “polypeptide” and this term embraces “peptides,” “peptidomimetics,” and “proteins.” “Peptides” are traditionally considered to range in size from about 4 to about 50 amino acids. Polypeptides larger than about 50 amino acids are generally termed “proteins.”

C5 inhibitor polypeptides may be linear or cyclic. Cyclic polypeptides include any polypeptides that have as part of their structure one or more cyclic features such as a loop and/or an internal linkage. In some embodiments, cyclic polypeptides are formed when a molecule acts as a bridging moiety to link two or more regions of the polypeptide. As used herein, the term “bridging moiety” refers to one or more components of a bridge formed between two adjacent or non-adjacent amino acids, non-natural amino acids or non-amino acids in a polypeptide. Bridging moieties may be of any size or composition. In some embodiments, bridging moieties may include one or more chemical bonds between two adjacent or non-adjacent amino acids, non-natural amino acids, non-amino acid residues or combinations thereof. In some embodiments, such chemical bonds may be between one or more functional groups on adjacent or non-adjacent amino acids, non-natural amino acids, non-amino acid residues or combinations thereof. Bridging moieties may include one or more of an amide bond (lactam), disulfide bond, thioether bond, aromatic ring, triazole ring, and hydrocarbon chain. In some embodiments, bridging moieties include an amide bond between an amine functionality and a carboxylate functionality, each present in an amino acid, non-natural amino acid or non-amino acid residue side chain. In some embodiments, the amine or carboxylate functionalities are part of a non-amino acid residue or non-natural amino acid residue.

C5 inhibitor polypeptides may be cyclized through the carboxy terminus, the amino terminus, or through any other convenient point of attachment, such as, for example, through the sulfur of a cysteine (e.g., through the formation of disulfide bonds between two cysteine residues in a sequence) or any side-chain of an amino acid residue. Further linkages forming cyclic loops may include, but are not limited to, maleimide linkages, amide linkages, ester linkages, ether linkages, thiol ether linkages, hydrazone linkages, or acetamide linkages.

In some embodiments, peptides may be synthesized on solid supports (e.g., rink amide resin) via solid phase peptide synthesis (SPPS). SPPS methods are known in the art and may be performed with orthogonal protecting groups. In some embodiments, peptides of the present disclosure may be synthesized via SPPS with Fmoc chemistry and/or Boc chemistry. Synthesized peptides may be cleaved from solid supports using standard techniques.

Peptides may be purified via chromatography [e.g., size exclusion chromatography (SEC) and/or high-performance liquid chromatography (HPLC)]. HPLC may include reverse phase HPLC (RP-HPLC). Peptides may be freeze-dried after purification. Purified peptides may be obtained as pure peptide or as a peptide salt. Residual salts making up peptide salts may include, but are not limited to, trifluoroacetic acid (TFA), acetate, and/or hydrochloride. In some embodiments, peptides of the present disclosure are obtained as peptide salts. The peptide salts may be peptide salts with TFA. Residual salts may be removed from purified peptides according to known methods (e.g., through use of desalting columns).

In some embodiments, cyclic C5 inhibitor polypeptides of the present disclosure are formed using a lactam moiety. Such cyclic polypeptides may be formed, for example, by synthesis on a solid support Wang resin using standard Fmoc chemistry. In some cases, Fmoc-ASP(allyl)-OH and Fmoc-LYS(alloc)-OH are incorporated into polypeptides to serve as precursor monomers for lactam bridge formation.

C5 inhibitor polypeptides of the present disclosure may be peptidomimetics. A “peptidomimetic” or “polypeptide mimetic” is a polypeptide in which the molecule contains structural elements that are not found in natural polypeptides (i.e., polypeptides comprised of only the 20 proteinogenic amino acids). In some embodiments, peptidomimetics are capable of recapitulating or mimicking the biological action(s) of a natural peptide. A peptidomimetic may differ in many ways from natural polypeptides, for example through changes in backbone structure or through the presence of amino acids that do not occur in nature. In some cases, peptidomimetics may include amino acids with side chains that are not found among the known 20 proteinogenic amino acids; non-polypeptide-based bridging moieties used to effect cyclization between the ends or internal portions of the molecule; substitutions of the amide bond hydrogen moiety by methyl groups (N-methylation) or other alkyl groups; replacement of a peptide bond with a chemical group or bond that is resistant to chemical or enzymatic treatments; N- and C-terminal modifications; and/or conjugation with a non-peptidic extension (such as polyethylene glycol, lipids, carbohydrates, nucleosides, nucleotides, nucleoside bases, various small molecules, or phosphate or sulfate groups).

As used herein, the term “amino acid” includes the residues of the natural amino acids as well as non-natural amino acids. The 20 natural proteinogenic amino acids are identified and referred to herein by either the one-letter or three-letter designations as follows: aspartic acid (Asp:D), isoleucine (Ile:I), threonine (Thr:T), leucine (Leu:L), serine (Ser:S), tyrosine (Tyr:Y), glutamic acid (Glu:E), phenylalanine (Phe:F), proline (Pro:P), histidine (His:H), glycine (Gly:G), lysine (Lys:K), alanine (Ala:A), arginine (Arg:R), cysteine (Cys:C), tryptophan (Trp:W), valine (Val:V), glutamine (Gln:Q) methionine (Met:M), asparagine (Asn:N). Naturally occurring amino acids exist in their levorotary (L) stereoisomeric forms. Amino acids referred to herein are L-stereoisomers except where otherwise indicated. The term “amino acid” also includes amino acids bearing a conventional amino protecting group (e.g. acetyl or benzyloxycarbonyl), as well as natural and non-natural amino acids protected at the carboxy terminus (e.g., as a (C1-C6) alkyl, phenyl or benzyl ester or amide; or as an alpha-methylbenzyl amide). Other suitable amino and carboxy protecting groups are known to those skilled in the art (See for example, Greene, T. W.: Wutz, P. G. M., Protecting Groups In Organic Synthesis; second edition, 1991, New York, John Wiley & sons, Inc., and documents cited therein, the contents of each of which are herein incorporated by reference in their entirety). Polypeptides and/or polypeptide compositions of the present disclosure may also include modified amino acids.

“Non-natural” amino acids have side chains or other features not present in the 20 naturally-occurring amino acids listed above and include, but are not limited to: N-methyl amino acids. N-alkyl amino acids, alpha, alpha substituted amino acids, beta-amino acids, alpha-hydroxy amino acids, D-amino acids, and other non-natural amino acids known in the art (See, e.g., Josephson et al., (2005) J. Am. Chem. Soc. 127: 11727-11735; Forster, A. C. et al. (2003) Proc. Natl. Acad. Sci. USA 100: 6353-6357: Subtelny et al., (2008) J. Am. Chem. Soc. 130: 6131-6136: Hartman, M. C. T. et al. (2007) PLoS ONE 2:e972; and Hartman et al., (2006) Proc. Natl. Acad. Sci. USA 103:4356-4361). Further non-natural amino acids useful for the optimization of polypeptides and/or polypeptide compositions of the present disclosure include, but are not limited to 1,2,3,4-tetrahydroisoquinoline-1-carboxylic acid, 1-amino-2,3-hydro-1H-indene-1-carboxylic acid, homolysine, homoarginine, homoserine, 2-aminoadipic acid, 3-aminoadipic acid, beta-alanine, aminopropionic acid, 2-aminobutyric acid, 4-aminobutyric acid, 5-aminopentanoic acid, 5-aminohexanoic acid, 6-aminocaproic acid, 2-aminoheptanoic acid, 2-aminoisobutyric acid, 3-aminoisobutyric acid, 2-aminopimelic acid, desmosine, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylpentylglycine, naphthylalanine, ornithine, pentylglycine, thioproline, norvaline, tert-butylglycine, phenylglycine, azatryptophan, 5-azatryptophan, 7-azatryptophan, 4-fluorophenylalanine, penicillamine, sarcosine, homocysteine, 1-aminocyclopropanccarboxylic acid, 1-aminocyclobutanecarboxylic acid, 1-aminocyclopentanecarboxylic acid, I-aminocyclohexanecarboxylic acid, 4-aminotetrahydro-2H-pyran-4-carboxylic acid, (S)-2-amino-3-(1H-tetrazol-5-yl)propanoic acid, cyclopentylglycine, cyclohexylglycine, cyclopropylglycine, η-ω-methyl-arginine, 4-chlorophenylalanine, 3-chlorotyrosine, 3-fluorotyrosine, 5-fluorotryptophan, 5-chlorotryptophan, citrulline, 4-chloro-homophenylalanine, homophenylalanine, 4-aminomethyl-phenylalanine, 3-aminomethyl-phenylalanine, octylglycine, norleucine, tranexamic acid, 2-amino pentanoic acid, 2-amino hexanoic acid, 2-amino heptanoic acid, 2-amino octanoic acid, 2-amino nonanoic acid, 2-amino decanoic acid, 2-amino undecanoic acid, 2-amino dodecanoic acid, aminovaleric acid, and 2-(2-aminoethoxy)acetic acid, pipecolic acid, 2-carboxy azetidine, hexafluoroleucine, 3-Fluorovaline, 2-amino-4,4-difluoro-3-methylbutanoic acid, 3-fluoro-isoleucine, 4-fluoroisoleucine, 5-fluoroisoleucine, 4-methyl-phenylglycine, 4-ethyl-phenylglycine, 4-isopropyl-phenylglycine, (S)-2-amino-5-azidopentanoic acid (also referred to herein as “X02”), (S)-2-aminohept-6-enoic acid (also referred to herein as “X30”), (S)-2-aminopent-4-ynoic acid (also referred to herein as “X31”), (S)-2-aminopent-4-enoic acid (also referred to herein as “X12”), (S)-2-amino-5-(3-methylguanidino) pentanoic acid, (S)-2-amino-3-(4-(aminomethyl)phenyl)propanoic acid, (S)-2-amino-3-(3-(aminomethyl)phenyl)propanoic acid, (S)-2-amino-4-(2-aminobenzo[d]oxazol-5-yl)butanoic acid, (S)-leucinol, (S)-valinol, (S)-tert-leucinol, (R)-3-methylbutan-2-amine, (S)-2-methyl-1-phenylpropan-1-amine, and (S)—N,2-dimethyl-1-(pyridin-2-yl)propan-1-amine, (S)-2-amino-3-(oxazol-2-yl)propanoic acid, (S)-2-amino-3-(oxazol-5-yl)propanoic acid, (S)-2-amino-3-(1,3,4-oxadiazol-2-yl)propanoic acid, (S)-2-amino-3-(1,2,4-oxadiazol-3-yl)propanoic acid, (S)-2-amino-3-(5-fluoro-1H-indazol-3-yl)propanoic acid, and (S)-2-amino-3-(1H-indazol-3-yl)propanoic acid, (S)-2-amino-3-(oxazol-2-yl)butanoic acid, (S)-2-amino-3-(oxazol-5-yl) butanoic acid, (S)-2-amino-3-(1,3,4-oxadiazol-2-yl) butanoic acid, (S)-2-amino-3-(1,2,4-oxadiazol-3-yl) butanoic acid, (S)-2-amino-3-(5-fluoro-1H-indazol-3-yl) butanoic acid, and (S)-2-amino-3-(1H-indazol-3-yl) butanoic acid, 2-(2′MeOphenyl)-2-amino acetic acid, tetrahydro 3-isoquinolinecarboxylic acid and stereoisomers thereof (including, but not limited, to D and L isomers).

Additional non-natural amino acids that are useful in the optimization of polypeptides or polypeptide compositions of the present disclosure include but are not limited to fluorinated amino acids wherein one or more carbon bound hydrogen atoms are replaced by fluorine. The number of fluorine atoms included can range from 1 up to and including all of the hydrogen atoms. Examples of such amino acids include but are not limited to 3-fluoroproline, 3,3-difluoroproline, 4-fluoroproline, 4,4-difluoroproline, 3,4-difluroproline, 3,3,4,4-tetrafluoroproline, 4-fluorotryptophan, 5-flurotryptophan, 6-fluorotryptophan, 7-fluorotryptophan, and stereoisomers thereof.

Further non-natural amino acids that are useful in the optimization of polypeptides of the present disclosure include but are not limited to those that are disubstituted at the α-carbon. These include amino acids in which the two substituents on the α-carbon are the same, for example α-amino isobutyric acid, and 2-amino-2-ethyl butanoic acid, as well as those where the substituents are different, for example α-methylphenylglycine and α-methylproline. Further the substituents on the α-carbon may be taken together to form a ring, for example 1-aminocyclopentanecarboxylic acid, 1-aminocyclobutanecarboxylic acid, 1-aminocyclohexanecarboxylic acid, 3-aminotetrahydrofuran-3-carboxylic acid, 3-aminotetrahydropyran-3-carboxylic acid, 4-aminotetrahydropyran-4-carboxylic acid, 3-aminopyrrolidine-3-carboxylic acid, 3-aminopiperidine-3-carboxylic acid, 4-aminopiperidine-4-carboxylic acid, and stereoisomers thereof.

Additional non-natural amino acids that are useful in the optimization of polypeptides or polypeptide compositions of the present disclosure include but are not limited to analogs of tryptophan in which the indole ring system is replaced by another 9 or 10 membered bicyclic ring system with 0, 1, 2, 3 or 4 heteroatoms independently selected from N, O, or S. Each ring system may be saturated, partially unsaturated, or fully unsaturated. The ring system may be substituted by 0, 1, 2, 3, or 4 substituents at any substitutable atom. Each substituent may be independently selected from H, F, Cl, Br, CN, COOR, CONRR′, oxo, OR. NRR′. Each Rand R′ may be independently selected from H, C1-C20 alkyl, or C1-C20 alkyl-O—C1-20 alkyl.

In some embodiments, analogs of tryptophan (also referred to herein as “tryptophan analogs”) may be useful in the optimization of polypeptides or polypeptide compositions of the present disclosure. Tryptophan analogs may include, but are not limited to, 5-fluorotryptophan [(5-F)W], 5-methyl-O-tryptophan [(5-MeO)W], 1-methyltryptophan [(1-Me-W) or (1-Me)W]. D-tryptophan (D-Trp), azatryptophan (including, but not limited to 4-azatryptophan, 7-azatryptophan and 5-azatryptophan,) 5-chlorotryptophan, 4-fluorotryptophan, 6-fluorotryptophan, 7-fluorotryptophan, and stereoisomers thereof. Except where indicated to the contrary, the term “azatryptophan” and its abbreviation, “azaTrp,” as used herein, refer to 7-azatryptophan.

Modified amino acid residues useful for the optimization of polypeptides and/or polypeptide compositions of the present disclosure include, but are not limited to those which are chemically blocked (reversibly or irreversibly); chemically modified on their N-terminal amino group or their side chain groups; chemically modified in the amide backbone, as for example, N-methylated, D (non-natural amino acids) and L (natural amino acids) stereoisomers; or residues wherein the side chain functional groups are chemically modified to another functional group. In some embodiments, modified amino acids include without limitation, methionine sulfoxide; methionine sulfone; aspartic acid-(beta-methyl ester), a modified amino acid of aspartic acid; N-ethylglycine, a modified amino acid of glycine; alanine carboxamide; and/or a modified amino acid of alanine. Non-natural amino acids may be purchased from Sigma-Aldrich (St. Louis, Mo.), Bachem (Torrance, Calif.) or other suppliers. Non-natural amino acids may further include any of those listed in Table 2 of US patent publication US 2011/0172126, the contents of which are incorporated herein by reference in their entirety.

The present disclosure contemplates variants and derivatives of polypeptides presented herein. These include substitutional, insertional, deletional, and covalent variants and derivatives. As used herein, the term “derivative” is used synonymously with the term “variant” and refers to a molecule that has been modified or changed in any way relative to a reference molecule or starting molecule.

Polypeptides of the present disclosure may include any of the following components, features, or moieties, for which abbreviations used herein include: “Ac” and “NH2” indicate acetyl and amidated termini, respectively; “Nvl” stands for norvaline; “Phg” stands for phenylglycine; “Tbg” stands for tert-butylglycine; “Chg” stands for cyclohexylglycine; “(N-Me)X” stands for the N-methylated form of the amino acid indicated by the letter or three letter amino acid code in place of variable “X” written as N-methyl-X [e.g. (N-Me)D or (N-Me)Asp stand for the N-methylated form of aspartic acid or N-methyl-aspartic acid]: “azaTrp” stands for azatryptophan; “(4-F)Phe” stands for 4-fluorophenylalanine; “Tyr(OMe)” stands for O-methyl tyrosine, “Aib” stands for amino isobutyric acid; “(homo)F” or “(homo)Phe” stands for homophenylalanine; “(2-OMe)Phg” refers to 2-O-methylphenylglycine; “(5-F)W” refers to 5-fluorotryptophan; “D-X” refers to the D-stereoisomer of the given amino acid “X” [e.g. (D-Chg) stands for D-cyclohexylglycine]: “(5-MeO)W” refers to 5-methyl-O-tryptophan; “homoC” refers to homocysteine; “(1-Me-W)” or “(1-Me)W” refers to 1-methyltryptophan; “Nle” refers to norleucine; “Tiq” refers to a tetrahydroisoquinoline residue; “Asp(T)” refers to (S)-2-amino-3-(1H-tetrazol-5-yl)propanoic acid; “(3-Cl-Phe)” refers to 3-chlorophenylalanine: “[(N-Me-4-F)Phe]” or “(N-Me-4-F)Phe” refers to N-methyl-4-fluorophenylalanine; “(m-Cl-homo)Phe” refers to meta-chloro homophenylalanine; “(des-amino)C” refers to 3-thiopropionic acid: “(alpha-methyl)D” refers to alpha-methyl L-aspartic acid; “2Nal” refers to 2-naphthylalanine: “(3-aminomethyl)Phe” refers to 3-aminomethyl-L-phenyalanine; “Cle” refers to cycloleucine; “Ac-Pyran” refers to 4-amino-tetrahydro-pyran-4-carboxylic acid: “(Lys-C16)” refers to N-ε-palmitoyl lysine: “(Lys-C12)” refers to N-ε-lauryl lysine: “(Lys-C10)” refers to N-ε-capryl lysine; “(Lys-C8)” refers to N-ε-caprylic lysine; “[xXylyl(y, z)]” refers to the xylyl bridging moiety between two thiol containing amino acids where x may be m, p or o to indicate the use of meta-, para- or ortho-dibromoxylenes (respectively) to generate bridging moieties and the numerical identifiers, y and z, place the amino acid position within the polypeptide of the amino acids participating in the cyclization; “[cyclo(y,z)]” refers to the formation of a bond between two amino acid residues where the numerical identifiers, y and z, place the position of the residues participating in the bond: “[cyclo-olefinyl(y,z)]” refers to the formation of a bond between two amino acid residues by olefin metathesis where the numerical identifiers, y and z, place the position of the residues participating in the bond; “[cyclo-thioalkyl(y,z)]” refers to the formation of a thioether bond between two amino acid residues where the numerical identifiers, y and z, place the position of the residues participating in the bond; “[cyclo-triazolyl(y,z)]” refers to the formation of a triazole ring between two amino acid residues where the numerical identifiers, y and z, place the position of the residues participating in the bond. “B20” refers to N-ε-(PEG2-γ-glutamic acid-N-α-octadecanedioic acid) lysine [also known as (1S,28S)-1-amino-7,16,25,30-tetraoxo-9,12,18,21-tetraoxa-6,15,24,29-tetraazahexatetracontane-1,28,46-tricarboxylic acid.]

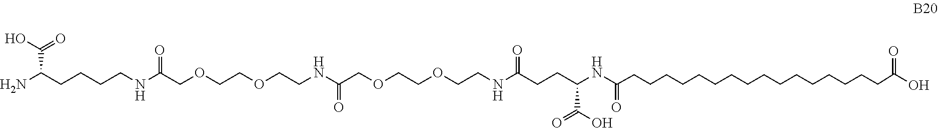 [SMILES: N[C@@H](CCCCNC(=O)COCCOCCNC(=O)COCCOCCNC(=O)CC[C@H](NC(=O)CCCCCCCCCCCCCCCCC(=O)O)C(=O)O)C(=O)O]

“B28” refers to N-ε-(PEG24-γ-glutamic acid-N-α-hexadecanoyl)lysine.

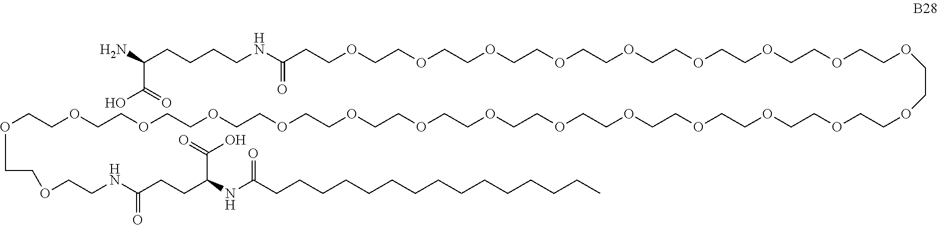 [SMILES: CCCCCCCCCCCCCCCC(=O)N[C@@H](CCC(=O)NCCOCCOCCOCCOCCOCCOCCOCCOCCOCCOCCOCCOCCOCCOCCOCCOCCOCCOCCOCCOCCOCCOCCOCCOCCC(=O)NCCCC[C@H](N)C(=O)O)C(=O)O]

“K14” refers to N-ε-1-(4,4-dimethyl-2,6-dioxocyclohex-1-ylidene)-3-methylbutyl-L-lysine. All other symbols refer to the standard one-letter amino acid code.

Some C5 inhibitor polypeptides include from about 5 amino acids to about 10 amino acids, from about 6 amino acids to about 12 amino acids, from about 7 amino acids to about 14 amino acids, from about 8 amino acids to about 16 amino acids, from about 10 amino acids to about 18 amino acids, from about 12 amino acids to about 24 amino acids, or from about 15 amino acids to about 30 amino acids. In some cases, C5 inhibitor polypeptides include at least 10 amino acids. In some cases, C5 inhibitor polypeptides include at least 30 amino acids. C5 inhibitor polypeptides may include 14, 15 or 16 amino acids, (e.g., 15 amino acids).

Some C5 inhibitors of the present disclosure include a C-terminal lipid moiety. Such lipid moieties may include fatty acyl groups (e.g., saturated or unsaturated fatty acyl groups). In some cases, the fatty acyl group may be a palmitoyl group.

C5 inhibitors having fatty acyl groups may include one or more molecular linkers joining the fatty acids to the peptide. Such molecular linkers may include amino acid residues. In some cases, L-γ glutamic acid residues may be used as molecular linkers. In some cases, molecular linkers may include one or more polyethylene glycol (PEG) linkers. PEG linkers of the present disclosure may include from about 1 to about 5, from about 2 to about 10, from about 4 to about 20, from about 6 to about 24, from about 8 to about 32, or at least 32 PEG units.

C5 inhibitors disclosed herein may have molecular weights of from about 200 g/mol to about 600 g/mol, from about 500 g/mol to about 2000 g/mol, from about 1000 g/mol to about 5000 g/mol, from about 3000 g/mol to about 4000 g/mol, from about 2500 g/mol to about 7500 g/mol, from about 5000 g/mol to about 10000 g/mol, or at least 10000 g/mol.

In some embodiments, C5 inhibitor polypeptides of the present disclosure include zilucoplan. The core amino acid sequence of zilucoplan ([cyclo(1,6)]Ac—K-V-E-R-F-D-(N-Me)D-Tbg-Y-azaTrp-E-Y—P-Chg-K; SEQ ID NO: 1) includes 15 amino acids (all L-amino acids), including 4 non-natural amino acids [N-methyl-aspartic acid or “(N-Me)D”, tert-butyiglycine or “Tbg”, 7-azatryptophan or “azaTrp”, and cyclohexylglycine or “Chg” ]; a lactam bridge between K1 and D6 of the polypeptide sequence; and a C-terminal lysine reside with a modified side chain, forming a N-(PEG24-γ-glutamic acid-N-α-hexadecanoyl)lysine residue (also referred to herein as “B28”). The C-terminal lysine side chain modification includes a polyethyleneglycol (PEG) spacer (PEG24), with the PEG24 being attached to an L-γ glutamic acid residue that is derivatized with a palmitoyl group.

The free acid form of zilucoplan has a molecular formula of C₁₇₂H₂₇₈N₂₄O₅₅, a molecular weight of 3562.23 Daltons (Da), and an exact mass of 3559.97 amu (see CAS Number 1841136-73-9). The tetra sodium form of zilucoplan has a molecular formula of C₁₁₂H₂₇₈N₂₄O₅₅Na₄. The chemical structure of sodium salt form of zilucoplan is shown in structure I:

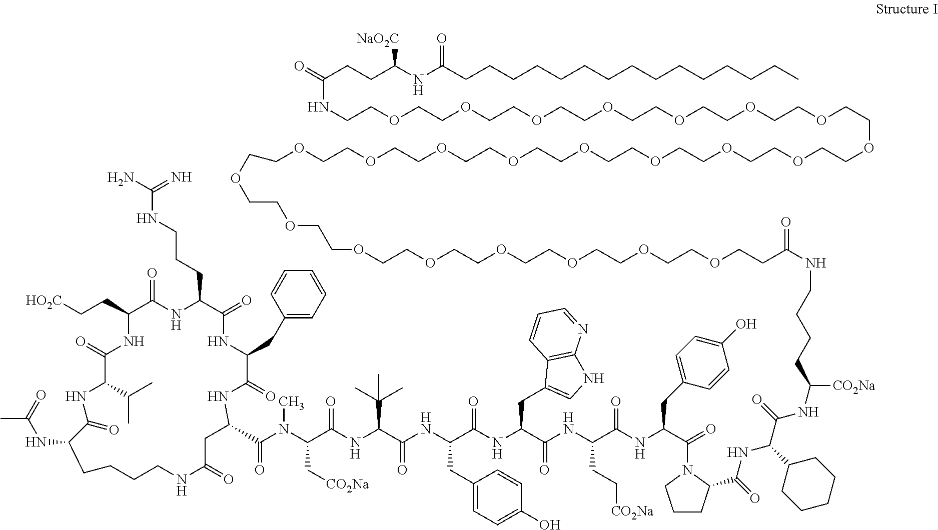

The four sodium ions in the structure are shown associated with designated carboxylates, but they may be associated with any of the acidic groups in the molecule. The zilucoplan drug substance is typically provided as the sodium salt form and is lyophilized. The free base form of zilucoplan or any pharmaceutically acceptable salt of zilucoplan are encompassed by the term “zilucoplan”.

In some embodiments, the C5 inhibitors of the present disclosure include variants of zilucoplan. Herein, references to zilucoplan include active metabolites or variants thereof, i.e., active metabolites or variants with C5 inhibiting activity. In some zilucoplan variants, the C-terminal lysine side chain moiety may be altered. In some cases, the PEG24 spacer (having 24 PEG subunits) of the C-terminal lysine side chain moiety may include fewer or additional PEG subunits. In other cases, the palmitoyl group of the C-terminal lysine side chain moiety may be substituted with another saturated or unsaturated fatty acid. In further cases, the L-γ glutamic acid linker of the C-terminal lysine side chain moiety (between PEG and acyl groups) may be substituted with an alternative amino acid or non-amino acid linker.

In some embodiments, C5 inhibitors may include active metabolites or variants of zilucoplan. Metabolites may include co-hydroxylation of the palmitoyl tail. Such variants may be synthesized or may be formed by hydroxylation of a zilucoplan precursor.

In some embodiments, zilucoplan variants may include modifications to the core polypeptide sequence in zilucoplan that may be used in combination with one or more of the cyclic or C-terminal lysine side chain moiety features of zilucoplan. Such variants may have at least 50%, at least 55%, at least 65%, at least 70%, at least 80%, at least 85%, at least 90%, or at least 95% sequence identity to the core polypeptide sequence of (SEQ ID NO: 1).

In some cases, zilucoplan variants may be cyclized by forming lactam bridges between amino acids other than those used in zilucoplan.

In some embodiments, C5 inhibitors of the present disclosure may include any of those listed in Table 1 of United States Publication Number US 2017/0137468, the contents of which are herein incorporated by reference in their entirety.

C5 inhibitors of the present disclosure may be developed or modified to achieve specific binding characteristics. Inhibitor binding may be assessed by determining rates of association and/or dissociation with a particular target. In some cases, compounds demonstrate strong and rapid association with a target combined with a slow rate of dissociation. In some embodiments, C5 inhibitors of the present disclosure demonstrate strong and rapid association with C5. Such inhibitors may further demonstrate slow rates of dissociation with C5.

C5 protein-binding C5 inhibitors disclosed herein, may bind to C5 complement protein with an equilibrium dissociation constant (K_{D}) of from about 0.001 nM to about 0.01 nM, from about 0.005 nM to about 0.05 nM, from about 0.01 nM to about 0.1 nM, from about 0.05 nM to about 0.5 nM, from about 0.1 nM to about 1.0 nM, from about 0.5 nM to about 5.0 nM, from about 2 nM to about 10 nM, from about 8 nM to about 20 nM, from about 15 nM to about 45 nM, from about 30 nM to about 60 nM, from about 40 nM to about 80 nM, from about 50 nM to about 100 nM, from about 75 nM to about 150 nM, from about 100 nM to about 500 nM, from about 200 nM to about 800 nM, from about 400 nM to about 1,000 nM or at least 1,000 nM.

In some embodiments, C5 inhibitors of the present disclosure block the formation or generation of C5a from C5. In some case, formation or generation of C5a is blocked following activation of the alternative pathway of complement activation. In some cases, C5 inhibitors of the present disclosure block the formation of the membrane attack complex (MAC). Such MAC formation inhibition may be due to C5 inhibitor binding to C5b subunits. C5 inhibitor binding to C5b subunits may prevent C6 binding, resulting in blockage of MAC formation. In some embodiments, this MAC formation inhibition occurs after activation of the classical, alternative, or lectin pathways.

C5 inhibitors of the present disclosure may be synthesized using chemical processes. In some cases, such synthesis eliminates risks associated with the manufacture of biological products in mammalian cell lines. In some cases, chemical synthesis may be simpler and more cost-effective than biological production processes.

In some embodiments, C5 inhibitor (e.g., zilucoplan and/or an active metabolite or variant thereof) compositions may be pharmaceutical compositions that include at least one pharmaceutically acceptable excipient. In some embodiments, the pharmaceutically acceptable excipient may include at least one of a salt and a buffering agent. The salt may be sodium chloride. The buffering agent may be sodium phosphate. Sodium chloride may be present at a concentration of from about 0.1 mM to about 1000 mM. In some cases, sodium chloride may be present at a concentration of from about 25 mM to about 100 mM. Sodium phosphate may be present at a concentration of from about 0.1 mM to about 1000 mM. In some cases, sodium phosphate is present at a concentration of from about 10 mM to about 100 mM.

In some embodiments. C5 inhibitor (e.g., zilucoplan and/or an active metabolite or variant thereof) compositions may include from about 0.01 mg/mL to about 4000 mg/mL of a C5 inhibitor. In some cases, C5 inhibitors are present at a concentration of from about 1 mg/mL to about 400 mg/mL.

Polypeptide-based C5 inhibitors (e.g., zilucoplan and/or active metabolites or variants thereof) may be used to treat indications benefiting from rapid and/or enhanced inhibitor tissue distribution. The tissue may include muscle and/or neuromuscular junction (NMJ). Polypeptide inhibitors (e.g., zilucoplan) may provide superior penetration into muscle and/or NMJ compared to antibodies based on smaller size and/or favorable charge profile. Such penetration may lead to faster relief from overactive complement. Further, polypeptide inhibitor (e.g., zilucoplan) penetration may stabilize and/or improve NMJ membrane potential by preventing MAC pore formation. Accordingly, safety factor at the NMJ may be improved. The term “safety factor” refers to excess transmitter levels released after nerve impulse that ensure neuromuscular transmission effectiveness under physiological stress. The excess is the amount beyond that required to trigger muscle fiber action potential and contributes to membrane potential restoration.

### Isotopic Variations

Compounds of the present disclosure may include one or more atoms that are isotopes. As used herein, the term “isotope” refers to a chemical element that has one or more additional neutrons. In some embodiments, compounds of the present disclosure may be deuterated. As used herein, the term “deuterated” refers to a substance that has had one or more hydrogen atoms replaced by deuterium isotopes. Deuterium isotopes are isotopes of hydrogen. The nucleus of hydrogen contains one proton while deuterium nuclei contain both a proton and a neutron. Compounds and compositions of the present disclosure may be deuterated in order to change a physical property, such as stability, or to allow for use in diagnostic and experimental applications.

### II. Methods

In some embodiments, the present disclosure provides methods related to using and evaluating compounds and compositions for therapeutic treatment of neurological disorders, such as MG. Some methods include modulating complement activity using compounds and/or compositions described herein.

### Therapeutic Indications

In some embodiments, the present disclosure provides methods of treating therapeutic indications using compounds and compositions described herein. A “therapeutic indication,” as used herein, refers to any disease, disorder, condition, or symptom that may be alleviated, cured, improved, reversed, stabilized, or otherwise addressed through one or more forms of therapeutic intervention (e.g., therapeutic agent administration or specific treatment method).

Therapeutic indications may include complement-related indications. As used herein, the term “complement-related indication” refers to any disease, disorder, condition, or symptom related to the complement system, e.g., cleavage or processing of a complement component, such as C5. In some embodiments, methods of the present disclosure include treating complement-related indications with compounds and compositions presented herein.

In some embodiments, methods of the disclosure include treating complement-related indications by inhibiting complement activity in a subject using compounds and compositions presented herein. In some cases, the percentage of complement activity inhibited in a subject may be at least 10%, at least 20%, at least 30%, at least 40%, at least 50%, at least 60%, at least 70%, at least 80%, at least, 85%, at least 90%, at least 95%, at least 96%, at least 97%, at least 98%, at least 99%, at least 99.5%, or at least 99.9%. In some cases, this level of inhibition and/or maximum inhibition of complement activity may be achieved by from about 1 hour after an administration to about 3 hours after an administration, from about 2 hours after an administration to about 4 hours after an administration, from about 3 hours after an administration to about 10 hours after an administration, from about 5 hours after an administration to about 20 hour after an administration, or from about 12 hours after an administration to about 24 hours after an administration. Inhibition of complement activity may continue throughout a period of at least 1 day, of at least 2 days, of at least 3 days, of at least 4 days, of at least 5 days, of at least 6 days, of at least 7 days, of at least 2 weeks, of at least 3 weeks, or at least 4 weeks. In some cases, this level of inhibition may be achieved through daily administration. Such daily administration may include administration for at least 2 days, for at least 3 days, for at least 4 days, for at least 5 days, for at least 6 days, for at least 7 days, for at least 2 weeks, for at least 3 weeks, for at least 4 weeks, for at least 2 months, for at least 4 months, for at least 6 months, for at least 1 year, or for at least 5 years. In some cases, subjects may be administered compounds or compositions of the present disclosure for the life of such subjects.

In some embodiments, the present disclosure provides methods of treating complement-related indications by inhibiting C5 activity in a subject. “C5-dependent complement activity” or “C5 activity,” as used herein refers to activation of the complement cascade through cleavage of C5, the assembly of downstream cleavage products of C5, or any other process or event attendant to, or resulting from, the cleavage of C5. In some cases, the percentage of C5 activity inhibited in a subject may be at least 10%, at least 20%, at least 30%, at least 40%, at least 50%, at least 60%, at least 70%, at least 80%, at least, 85%, at least 90%, at least 95%, at least 96%, at least 97%, at least 98%, at least 99%, at least 99.5%, or at least 99.9%.

C5 inhibitors may be used to treat one or more complement-related indications, wherein few or no adverse effects result from treatment. In some cases, no adverse cardiovascular, respiratory, and/or central nervous system (CNS) effects occur. In some cases, no changes in heart rate and/or arterial blood pressure occur. In some cases, no changes to respiratory rate, tidal volume, and/or minute volume occur.

By “lower” or “reduce” in the context of a disease marker or symptom is meant a significant decrease in such level, often statistically significant. The decrease can be, for example, at least 10%, at least 20%, at least 30%, at least 40% or more, and is preferably down to a level accepted as within the range of normal for an individual without such disorder.

By “increase” or “raise” in the context of a disease marker or symptom is meant a significant rise in such level, often statistically significant. The increase can be, for example, at least 10%, at least 20%, at least 30%, at least 40% or more, and may be up to a level accepted as within the range of normal for an individual without such disorder.

A treatment or preventive effect is evident when there is a significant improvement, often statistically significant, in one or more parameters of disease status, or by a failure to worsen or to develop symptoms where they would otherwise be anticipated. As an example, a favorable change of at least 10% in a measurable parameter of disease, and at least 20%, 30%, 40%, 50% or more can be indicative of effective treatment. Efficacy for a given compound or composition can also be judged using an experimental animal model for the given disease as known in the art. When using an experimental animal model, efficacy of treatment is evidenced when a statistically significant modulation in a marker or symptom is observed.

Compounds of the present disclosure and additional therapeutic agents can be administered in combination. Such combinations may be in the same composition, or the additional therapeutic agents can be administered as part of a separate composition or by another method described herein.

In some embodiments, the present disclosure provides methods of inhibiting C5 activity in a tissue by contacting the tissue with a tissue-penetrating C5 inhibitor. As used herein, the term “tissue-penetrating” refers to a property characterized by tissue permeability. Agents with enhanced tissue-penetration may demonstrate better distribution in tissues when compared to agents with less or no tissue-penetration. Tissue penetration may be assessed by ability to cross basement membranes. As used herein, the term “basement membrane” refers to an extracellular matrix (ECM) protein layer separating endothelial cells from underlying tissues. Tissue penetration assessments may be done in vivo or in vitro and may include the use of basement membrane models. Such models may include measuring compound diffusion across artificial basement membranes. Such models may include the use of upper and lower reservoirs separated by an artificial basement membrane. Artificial basement membranes may include any of the ECM gel membranes described in Arends, F. et al. 2016. IntechOpen, DOI: 10.5772/62519, the contents of which are herein incorporated by reference in their entirety. ECM gel membranes may be prepared to include matrix components mimicking those found in the basal lamina of neuromuscular junctions. In some models, compounds being tested are introduced to upper reservoirs and compound diffusion is detected in lower reservoirs.

Tissue penetration assessment may include visual assessments e.g., through use of fluorescent labels to visualize analyte movement across basement membranes. Some assessments may include biochemical analysis of samples obtained from the penetrated side of a basement membrane.

In some embodiments, compound permeability may be determined using quantitative whole body analysis (QWBA). QWBA is a form of analysis that uses radiography to assess distribution of radiolabeled analytes. In some embodiments, radiolabeled compounds are administered to subjects and tissue distribution of the compounds is analyzed over time.

Tissue-penetrating C5 inhibitors may be polypeptides. Tissue-penetrating C5 inhibitors may include zilucoplan. Contacting tissues with the tissue-penetrating C5 inhibitors may include administering tissue-penetrating C5 inhibitors to tissues as part of a formulation. Such formulations may be administered by subcutaneous injection. Tissue-penetrating C5 inhibitors may be able to penetrate basement membranes. Basement membrane permeability of polypeptide tissue-penetrating C5 inhibitors may be greater than basement membrane permeability of larger proteins, such as antibodies. Such advantages may be due to restrictively large size of proteins and antibodies. Zilucoplan basement membrane permeability may be from about 3-fold to about 5-fold greater than basement membrane permeability of eculizumab, offering advantages over eculizumab for inhibiting C5 activity in tissues and treating related complement-related indications. In some embodiments, zilucoplan permeability enhances distribution in one or more of lung, heart, muscle, small intestine, large intestine, spleen, liver, bone, stomach, lymph node, fat, brain, pancreas, testes, and thymus, in comparison to eculizumab.

Polypeptide-based C5 inhibitors (e.g., zilucoplan and/or active metabolites or variants thereof) may be used to treat complement-related indications (e.g., myasthenia gravis) benefiting from rapid and/or enhanced inhibitor tissue distribution. The tissue may include muscle and/or neuromuscular junction (NMJ). Polypeptide inhibitors (e.g., zilucoplan) may provide superior penetration into muscle and/or NMJ compared to antibodies based on smaller size and/or favorable charge profile. Such penetration may lead to faster relief from overactive complement. Further, polypeptide inhibitor (e.g., zilucoplan) penetration may stabilize and/or improve NMJ membrane potential by preventing MAC pore formation. Accordingly, safety factor at the NMJ may be improved. The term “safety factor” refers to excess transmitter levels released after nerve impulse that ensure neuromuscular transmission effectiveness under physiological stress. The excess is the amount beyond that required to trigger muscle fiber action potential and contributes to membrane potential restoration.

In some embodiments, the present disclosure provides methods of treating complement-related indications in subjects by administering zilucoplan in combination with other therapeutic agents. Cyclosporine A is a known immunosuppressive agent, inhibitor of organic anion transporting polypeptide (OATP) 1B1 and OATP1B3, and is a potential comedication in PNH and other complement-related indications. In some embodiments, cyclosporine A and zilucoplan may be administered in combination to subjects with complement-related indications (e.g., myasthenia gravis). Cyclosporine A and zilucoplan may be administered in overlapping dosage regimens. Other immunosuppressive agents that may be administered in combination with or in overlapping dosage regiments with zilucoplan may include, but are not limited to, azathioprine, cyclosporine, mycophenolate mofetil, methotrexate, tacrolimus, cyclophosphamide, and rituximab.

In some embodiments, the present disclosure provides methods of treating complement-related indications in subjects by administering zilucoplan in combination with neonatal Fc receptor (FcRN) inhibitor treatments. FcRN inhibitor treatments may be used to treat autoimmune diseases that include autoantibody-mediated tissue destruction. FcRN inhibitor treatments may include intravenous immunoglobulin (IVIG) treatment, which reduces the half-life of IgG antibodies by overwhelming the Fc recycling mechanism with large doses of immunoglobulin. Some FcRN inhibitor treatments may include administration of DX-2504 or functionally equivalent variants thereof, e.g., DX-2507, which includes modifications to reduce aggregation and improve manufacturability (described in Nixon, A. E. et al. 2015. Front Immunol. 6:176). DX-2504 is an inhibitor of FcRN recycling. By inhibiting FcRN, DX-2504 inhibits Fc-mediated recycling, thereby reducing the half-life of IgG antibodies. Administration of DX-2504 may also be used in models of IVIG treatment. In some embodiments, zilucoplan may be administered to treat complement-related indications (e.g., myasthenia gravis) in overlapping dosage regimens with FcRN inhibitor treatments. The FcRN inhibitor treatments may include DX-2504 (or DX-2507) administration and/or IVIG treatment.

### Neurological Indications

In some embodiments, compounds and compositions disclosed herein may be used to treat complement-related indications that are neurological indications. A “neurological indication,” as used herein, refers to any disease, disorder, condition, or symptom related to the nervous system. In some embodiments, complement-related neurological indications include myasthenia gravis.

### Autoimmune Indications

In some embodiments, compounds and compositions disclosed herein may be used to treat complement-related indications that are autoimmune indications. As used herein, the term “autoimmune indication” refers to any disease, disorder, condition, or symptom related to self-destructive immune activity. The ability of the immune system to distinguish between self and non-self cells is a critical feature of this system. Pathology arises when the immune system is unable to make this distinction. The immune system may be divided into innate and adaptive systems, referring to nonspecific immediate defense mechanisms and more complex antigen-specific systems, respectively. The complement system is part of the innate immune system, recognizing and eliminating pathogens. Additionally, complement proteins may modulate adaptive immunity, connecting innate and adaptive responses. Autoimmune disease may involve certain tissues or organs of the body.

In the case of the complement system, vertebrate cells express inhibitory proteins that protect them from the effects of the complement cascade and this ensures that the complement system is directed against foreign pathogens. Many complement-related indications are associated with abnormal destruction of self-cells by the complement cascade.

In some embodiments, complement-related autoimmune indications include myasthenia gravis.

### Myasthenia Gravis

In some embodiments, compounds and compositions disclosed herein may be used to treat complement-related indications that include myasthenia gravis. Myasthenia gravis (MG) is a rare complement-mediated autoimmune disease characterized by the production of autoantibodies targeting proteins that are critical for the normal transmission of chemical or neurotransmitter signals from nerves to muscles, e.g., acetylcholine receptor (AChR) proteins. The presence of AChR autoantibodies in patient samples can be used as an indicator of disease. As used herein, the term “MG” embraces any form of MG. While about 15% of patients have symptoms that are confined to ocular muscles, the majority of patients experience generalized myasthenia gravis. As used herein, the term “*generalized myasthenia gravis” or “gMG” refers to MG that affects multiple muscle groups throughout the body. Although the prognosis of MG is generally benign, 10% to 15% of patients have refractory MG. As used herein, the term “refractory MG” or “rMG” refers to MG where disease control either cannot be achieved with current therapies, or results in severe side effects of immunosuppressive therapy. This severe form of MG affects approximately 9,000 individuals in the United States.

Patients with MG present with muscle weakness that characteristically becomes more severe with repeated use and recovers with rest. Muscle weakness can be localized to specific muscles, such as those responsible for eye movements, but often progresses to more diffuse muscle weakness. MG may even become life-threatening when muscle weakness involves the diaphragm and the other chest wall muscles responsible for breathing. This is the most feared complication of MG, known as myasthenic crisis or MG crisis, and requires hospitalization, intubation, and mechanical ventilation. Approximately 15% to 20% of patients with gMG experience a myasthenic crisis within two years of diagnosis.

The most common target of autoantibodies in MG is the acetylcholine receptor, or AChR, located at the neuromuscular junction, the point at which a motor neuron transmits signals to a skeletal muscle fiber. Current therapies for gMG focus on either augmenting the AChR signal or nonspecifically suppressing the autoimmune response. First-line therapy for symptomatic gMG is treatment with acetylcholinesterase inhibitors such as pyridostigmine, which is the only approved therapy for MG. Although sometimes adequate for control of mild ocular symptoms, pyridostigmine monotherapy is usually insufficient for the treatment of generalized weakness, and dosing of this therapy may be limited by cholinergic side effects. Therefore, in patients who remain symptomatic despite pyridostigmine therapy, corticosteroids with or without systemic immunosuppressives are indicated (Sanders D B, et al. 2016. Neurology. 87(4):419-25). Immunosuppressives used in gMG include azathioprine, cyclosporine, mycophenolate mofetil, methotrexate, tacrolimus, cyclophosphamide, and rituximab. To date, efficacy data for these agents are sparse and no steroidal or immunosuppressive therapy has been approved for the treatment of gMG. Moreover, all of these agents are associated with well-documented long-term toxicities. Surgical removal of the thymus may be recommended in patients with nonthymomatous gMG and moderate to severe symptoms in an effort to reduce the production of AChR autoantibodies (Wolfe G I, et al. 2016. N Engl J Med. 375(6):511-22). Intravenous (IV) immunoglobulin and plasma exchange (PLEX) are usually restricted to short-term use in patients with myasthenic crisis or life-threatening signs such as respiratory insufficiency or dysphagia (Sanders et al., 2016).

There is substantial evidence that supports the role of terminal complement cascade in the pathogenesis of AChR autoantibody-positive gMG. Results from animal models of experimental autoimmune MG have demonstrated that autoantibody immune complex formation at the neuromuscular junction triggers activation of the classical complement pathway, resulting in local activation of C3 and deposition of the membrane attack complex (MAC) at the neuromuscular junction, resulting in loss of signal transduction and eventual muscle weakness (Kusner L L, et al., 2012. Ann N Y Acad Sci. 1274(1):127-32).

In addition, inhibition of C5 has been validated as a target for the treatment of refractory gMG based on clinical studies with the C5-blocking antibody, eculizumab. Eculizumab is approved for use in MG and 2 other complement-driven rare diseases, paroxysmal nocturnal hemoglobinuria (PNH) and atypical hemolytic uremic syndrome (aHUS). In a Phase 2, randomized, double-blind, placebo-controlled trial, eculizumab was tested in 14 AChR autoantibody-positive patients with refractory gMG, who had a quantitative myasthenia gravis (QMG) score ≥12 and previously failed treatment with at least 2 immunosuppressant therapies (ISTs) (Howard, J F. 2013. Myasthenia Gravis Foundation of America. Clinical Overview of MG, the content of which is herein incorporated by reference in its entirety). Patients were randomized in a 1:1 ratio to receive either eculizumab or placebo. Patients on eculizumab received 600 mg per week for 4 weeks, followed by 900 mg every other week by IV infusion, for a total of 16 weeks of treatment. After a 5-week washout period, patients were crossed over to the opposite arm of the study. Patients who received placebo for the first 16 weeks of the study were treated with eculizumab and vice versa. The primary endpoints were safety and efficacy, as measured by the percentage of patients who achieved a ≥3-point reduction in QMG score. The impact of C5 inhibition by eculizumab in QMG score occurred rapidly (within 1 week of initiating treatment) and favored eculizumab compared with placebo across all study visits (p=0.0144). Following the initial 16-week treatment period, 6 out of 7 patients on eculizumab achieved a ≥3-point improvement in QMG score, compared with 4 out of 7 patients in the placebo arm. Of those patients who responded to eculizumab, 4 achieved an 8-point reduction in QMG score compared with only 1 in the placebo arm.

The QMG is a standardized and validated quantitative strength scoring system that was developed specifically for MG and has been used previously in clinical trials. The scoring system assesses 13 items relating to ocular, bulbar, and limb function (Bamet, C. et al. 2015. J Neuromuscul Dis. 2:301-11). Each item is scored from 0-3. Maximum total score is 39. Higher scores are representative of more severe impairment. Recent data suggest that improvements in the QMG score of 2 to 3 points may be considered clinically meaningful, depending upon disease severity [Barohn R J et al. 1998. Ann N Y Acad Sci. 841:769-772; Katzberg H D et al. 2014. Muscle Nerve. 49(5):661-665].

A Phase 3 trial (NCTO1997229) was also completed that enrolled 125 AChR autoantibody-positive patients with a Myasthenia Gravis-Activities of Daily Living (MG-ADL) score ≥6, who had previously failed 2 ISTs or had failed 1 IST and required chronic plasma exchange or IV immunoglobulin therapy. The MG-ADL is a brief 8-item survey designed to evaluate MG symptom severity. Each item is scored from 0-3. Maximum total score is 24. Higher scores are associated with more severe symptoms of MG. The MG-ADL has been shown to correlate with other validated MG outcome measures (e.g., MG-QOL15r), and a 2-point improvement in MG-ADL score is considered clinically meaningful [Wolfe G I et al. 1999. Neurology. 52(7):1487-9; Muppidi S et al. 2011. Muscle Nerve. 44(5):727-31]. The MG-QOL15r is a 15-item survey that was designed to assess quality of life in patients with MG based on patient reporting. Each item is scored from 0-2. Maximum total score is 30. Higher scores indicate more severe impact of the disease on aspects of the patient's life [Burns, T M et al. 2010. Muscle Nerve. 41(2):219-26; Burns T M et al. 2016. Muscle Nerve. 54(6):1015-22].

Patients were randomized 1:1 to receive either placebo or eculizumab for a 26-week treatment period, followed by an extension study. Patients receiving eculizumab were treated with 900 mg per week for 4 weeks followed by 1200 mg every other week by IV infusion. Eculizumab treatment was not associated with a statistically significant benefit relative to placebo in the primary endpoint of change from baseline in MG-ADL (p=0.0698) in this study. However, statistically significant results were observed in 18 of 22 prespecified analyses, including the secondary endpoint of change from baseline in QMG score (p=0.0129). Taken together, the results of these 2 clinical trials establish that inhibition of the terminal complement cascade by blocking cleavage of C5 is a clinically validated target for the treatment of gMG. Despite missing the primary endpoints in the Phase 3 trial, eculizumab was approved as a treatment for adult MG patients with AChR autoantibodies in the US, EU and Japan in 2017 based on the totality of data.

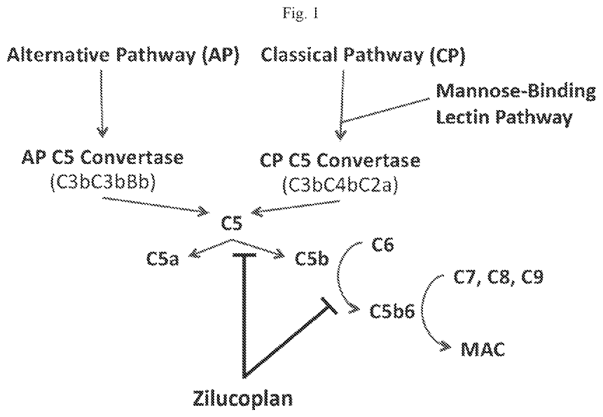

Binding of anti-AChR autoantibodies to the muscle endplate results in activation of the classical complement cascade and deposition of MAC on the post-synaptic muscle fiber leading to local damage to the muscle membrane, and reduced responsiveness of the muscle to stimulation by the neuron. Inhibition of terminal complement activity may be used to block complement-mediated damage resulting from MG (e.g., gMG and/or rMG). In some embodiments, C5 inhibitors disclosed herein may be used to treat MG. Such inhibitors may include zilucoplan. Inhibition of C5 cleavage may prevent downstream assembly and activity of the MAC, e.g., in post-junctional membranes of patient neuromuscular junctions, and reduce or prevent neuromuscular issues associated with MG (e.g., gMG and/or rMG). Unlike eculizumab, zilucoplan binds to the C5b portion of C5 and inhibits cleavage to C5a and C5b subunits. Zilucoplan also binds free C5b and prevents binding to C6 and subsequent MAC assembly. Accordingly, zilucoplan inhibits MAC assembly through two different mechanisms (see FIG. 1). Further, zilucoplan binds specifically to C5 and exhibits a strong and rapid association with C5, coupled with a slow dissociation rate.

### Screening

Subjects treated with zilucoplan may be screened prior to zilucoplan administration. As used herein, the term “screen” refers to a review or evaluation carried out for the purpose of selection or filtration. Subjects may be screened to select individuals in need of treatment. In some embodiments, subjects are screened to select individuals most likely to respond favorably to treatment. In some embodiments, screening is carried out to exclude individuals with greater risks associated with treatment. Screening may include assessment of QMG score. As described previously, the QMG is a standardized and validated quantitative strength scoring system that was developed specifically for MG and has been used previously in clinical trials. Higher scores are representative of more severe impairment. Recent data suggest that improvements in the QMG score of 2 to 3 points may be considered clinically meaningful, depending upon disease severity [Barohn R J et al. 1998. Ann N Y Acad Sci. 841:769-772: Katzberg H D et al. 2014. Muscle Nerve. 49(5):661-665, the contents of which are herein incorporated by reference in their entirety]. In some embodiments, subjects are screened to select subjects with QMG scores ≥12. In some embodiments, selected subjects have QMG scores with ≥4 QMG test items achieving a score of ≥2.

Subjects receiving MG therapies prior to or during screening may be maintained on such therapies during the screening process or may be required to withhold one or more treatments before or during the screening process. In some embodiments, a period of time between prior MG therapy and a screening assessment is required. The period of time may be required to obtain reliable results from a particular screening assessment. In some embodiments, subjects assessed for QMG score may be pulled from MG therapy for at least 10 hours prior to QMG score assessment. Subjects assessed for QMG score may be pulled from acetylcholinesterase inhibitor therapy (e.g., pyridostigmine treatment) for at least 10 hours prior to QMG score assessment.

Screening may include selecting subjects based on age. In some embodiments, screening may be carried out to select subjects with ages between 18 and 85 years old.

Screening may include selecting subjects previously diagnosed with gMG. The gMG diagnosis may be made according to Myasthenia Gravis Foundation of America (MGFA) criteria; Class II-IVa (see Howard, J. F., 2009. Myasthenia Gravis A Manual for the Health Care Provider, Myasthenia Gravis Foundation of America, Inc.).

Screening may include assessment of biomarker levels. In some embodiments, biomarkers include acetylcholinesterase receptor (AChR) autoantibody levels. AChR autoantibodies may lead to disease by binding AChR and stimulating complement activation. Accordingly, AChR autoantibody levels may be a good indicator of complement-mediated disease. In some embodiments, biomarkers include autoantibodies to muscle-specific tyrosine kinase (MuSK). Subjects with anti-MuSK antibodies are part of a distinct MG subset associated with less predictable treatment outcomes (Lavmic, D. et al. 2005. J Neurol Neurosurg Psychiatry. 76:1099-102). Screening may include excluding subjects with anti-MuSK antibodies from treatment and/or evaluations.

Screening may include review of subject prior and current treatments. In some embodiments, subjects are screened based on recent changes in treatments. In some embodiments, subjects are screened to confirm no change in corticosteroid dose or immunosuppressive therapy prior to screening. The screening may exclude subjects from treatment where subject corticosteroid treatment dose or immunosuppressive therapy regimen changes within the 30 days prior to screening.

Subjects may be screened for pregnancy status. In some embodiments, pregnant subjects may be excluded from treatment. Pregnancy status screening may be carried out by serum pregnancy test. In some embodiments, pregnancy screening may include urine pregnancy testing.

In some embodiments, screening may be carried out to identify subjects with a stage of MG that occurs prior to reaching a critical or crisis stage. Such screening may be carried out to identify subjects prior to developing MG or early in the disease process that may benefit from proactive or preventative treatment.

### Zilucoplan Treatment

Zilucoplan inhibits C5a formation in a dose-dependent manner upon activation of the classical pathway and inhibits C5b formation (as measured by C5b-9 or MAC deposition on a complement activating surface) upon activation of the classical and alternative complement pathways. (U.S. Pat. No. 9,937,222).

In some embodiments, methods of the present disclosure include methods of treating MG by zilucoplan administration to a subject. The MG treatment may include gMG. Zilucoplan administration may be subcutaneous (SC) administration. Zilucoplan may be administered at a dose of from about 0.01 mg/kg (mg zilucoplan/kg subject body weight) to about 1.0 mg/kg, from about 0.02 mg/kg to about 2.0 mg/kg, from about 0.05 mg/kg to about 3.0 mg/kg, from about 0.10 mg/kg to about 4.0 mg/kg, from about 0.15 mg/kg to about 4.5 mg/kg, from about 0.20 mg/kg to about 5.0 mg/kg, from about 0.30 mg/kg to about 7.5 mg/kg, from about 0.40 mg/kg to about 10 mg/kg, from about 0.50 mg/kg to about 12.5 mg/kg, from about 0.1 mg/kg to about 0.6 mg/kg, from about 1.0 mg/kg to about 15 mg/kg, from about 2.0 mg/kg to about 20 mg/kg, from about 5.0 mg/kg to about 25 mg/kg, from about 10 mg/kg to about 45 mg/kg, from about 20 mg/kg to about 55 mg/kg, from about 30 mg/kg to about 65 mg/kg, from about 40 mg/kg to about 75 mg/kg, from about 50 mg/kg to about 150 mg/kg, from about 100 mg/kg to about 250 mg/kg, from about 200 mg/kg to about 350 mg/kg, from about 300 mg/kg to about 450 mg/kg, from about 400 mg/kg to about 550 mg/kg, or from about 500 mg/kg to about 1000 mg/kg.

In some embodiments, zilucoplan may be administered at a dose of from about 0.10 mg/kg to about 0.42 mg/kg.

Methods of the present disclosure may include administering zilucoplan at a daily dose of from about 0.1 mg/kg to about 0.3 mg/kg. In some embodiments, zilucoplan is administered at a daily dose of 0.3 mg/kg. Subject QMG score and/or MG-ADL score may be reduced as a result of administration. QMG score may be reduced by 3 points by 8 weeks of treatment. MG-ADL score may be reduced by 2 points by 8 weeks of treatment. Risk of need for rescue therapy (IVIG or plasma exchange) may be reduced.

Zilucoplan administration may be by self-administration. Zilucoplan administration may include the use of prefilled syringes. Self-administration may include the use of self-administration devices. Self-administration devices may include or be incorporated with prefilled syringes.

Zilucoplan may be provided in solution. Zilucoplan solutions may include aqueous solutions. Zilucoplan solutions may include phosphate-buffered saline (PBS). Zilucoplan solutions may be preservative-free. Zilucoplan may be present in solutions at a concentration of from about 0.01 mg/mL to about 1 mg/mL, from about 0.05 mg/mL to about 2 mg/mL, from about 1 mg/mL to about 5 mg/mL, from about 2 mg/mL to about 10 mg/mL, from about 4 mg/mL to about 16 mg/mL, from about 5 mg/mL to about 20 mg/mL, from about 8 mg/mL to about 24 mg/mL, from about 10 mg/mL to about 30 mg/mL, from about 12 mg/mL to about 32 mg/mL, from about 14 mg/mL to about 34 mg/mL, from about 16 mg/mL to about 36 mg/mL, from about 18 mg/mL to about 38 mg/mL, from about 20 mg/mL to about 40 mg/mL, from about 22 mg/mL to about 42 mg/mL, from about 24 mg/mL to about 44 mg/mL, from about 26 mg/mL to about 46 mg/mL, from about 28 mg/mL to about 48 mg/mL, from about 30 mg/mL to about 50 mg/mL, from about 35 mg/mL to about 55 mg/mL, from about 40 mg/mL to about 60 mg/mL, from about 45 mg/mL to about 75 mg/mL, from about 50 mg/mL to about 100 mg/mL, from about 60 mg/mL to about 200 mg/mL, from about 70 mg/mL to about 300 mg/mL, from about 80 mg/mL to about 400 mg/mL, from about 90 mg/mL to about 500 mg/mL, or from about 100 mg/mL to about 1000 mg/mL.

In some embodiments, self-administration devices include zilucoplan solutions. Self-administration devices may include zilucoplan solution volumes of from about 0.010 mL to about 0.500 mL, from about 0.050 mL to about 0.600 mL, from about 0.100 mL to about 0.700 mL, from about 0.150 mL to about 0.810 mL, from about 0.200 mL to about 0.900 mL, from about 0.250 mL to about 1.00 mL, from about 0.300 mL to about 3.00 mL, from about 0.350 mL to about 3.50 mL, from about 0.400 mL to about 4.00 mL, from about 0.450 mL to about 4.50 mL, from about 0.500 mL to about 5.00 mL, from about 0.550 mL to about 10.0 mL, from about 0.600 mL to about 25.0 mL, from about 0.650 mL to about 50.0 mL, from about 0.700 mL to about 60.0 mL, from about 0.750 mL to about 75.0 mL, from about 0.800 mL to about 80.0 mL, from about 0.850 mL to about 85.0 mL, from about 0.900 mL to about 90.0 mL, from about 0.950 mL to about 95.0 mL, from about 1.00 mL to about 100 mL, from about 2.00 mL to about 200 mL, from about 5.00 mL to about 500 mL, from about 10.0 mL to about 750 mL, from about 25.0 mL to about 800 mL, from about 50.0 mL to about 900 mL, or from about 100 mL to about 1000 mL.

Zilucoplan treatment may be continuous or in one or more doses. In some embodiments, treatment is in doses that occur hourly, daily, bi-daily, weekly, bi-weekly, monthly, or combinations thereof. Zilucoplan treatment may include daily administration. Subject zilucoplan plasma levels may reach maximum concentration (Cₘₐₓ) on a first day of treatment. Serum hemolysis may be inhibited by zilucoplan treatment. In some embodiments, at least 90% hemolysis inhibition is achieved in subject serum with zilucoplan treatment. During administration, subjects may receive standard of care therapy for gMG. Standard of care therapies for MG may include, but are not limited to, plasma exchange, intravenous immunoglobin (IVIG) treatment, biologics (e.g., rituximab or eculizumab), pyridostigmine treatment, corticosteroid treatment, and/or immunosuppressive drug treatment. In some embodiments, subjects receive cholinesterase inhibitor treatment over the course of zilucoplan treatment.

Zilucoplan treatment for MG may be carried out with a variety of subjects from different demographic backgrounds and stages of disease. Treatment may be carried out with subjects with refractory (resistant or unresponsive to other standard therapies) or non-refractory MG. Refractory subjects may include those who have been resistant or unresponsive to prior therapy with eculizumab.

In some embodiments, subjects with a stage of MG that occurs prior to reaching a critical or crisis stage are treated with zilucoplan. Such treatment may be carried out to treat subjects prior to developing MG or early in the disease process to provide benefits of proactive or preventative treatment.

In some embodiments, the present invention provides zilucoplan for use in a method of treating MG comprising administering 0.1 to 0.3 mg/kg zilucoplan subcutaneously or intravenously to a subject. In some embodiments, the present invention provides zilucoplan for use in a method of treating MG comprising administering 0.1 mg/kg or 0.3 mg/kg zilucoplan subcutaneously or intravenously to the subject. In some embodiments, the present invention provides zilucoplan for use in a method of treating MG comprising administering 0.1 mg/kg or 0.3 mg/kg zilucoplan subcutaneously to the subject. In some embodiments, the present invention provides zilucoplan for use in a method of treating MG comprising administering 0.3 mg/kg zilucoplan subcutaneously to the subject. In some embodiments, the MG is gMG. In some embodiments, the subject is AChR autoantibody-positive.

### Evaluation

Subjects receiving zilucoplan treatment for MG may be evaluated for efficacy during or after treatment. As used herein, the term “treated subject” refers to an individual that has received at least one treatment. Zilucoplan treated subject evaluation may include evaluation of one or more metrics of efficacy. In some embodiments, evaluations may require subject treatments to be withheld for a period prior to evaluation. Some evaluations may require subjects to maintain consistent treatments before, during, and/or after evaluations. Withheld or maintained treatments may be zilucoplan treatments. In some embodiments, withheld or maintained treatments include other treatments for MG or for non-MG conditions.

Evaluations may be carried out to assess primary efficacy endpoints. As used herein, the term “primary endpoint” refers to a result that answers the most important inquiry addressed by a particular study. The term “secondary endpoint,” refers to a result that answers other relevant inquiries subordinate to a main inquiry. A primary efficacy endpoint is a result that addresses whether or not a treatment is effective, while a secondary efficacy endpoint addresses one or more peripheral inquiries (e.g., quality of life impact, side effect severity, etc.).

Evaluations may be carried out to assess subject MG characteristics. As used herein, the term “MG characteristic” refers to a physical or mental trait or set of traits associated with the presence of or severity of MG in a subject. MG characteristics may include scores obtained using different disease evaluation methods. MG characteristic may include, but are not limited to, QMG score, MG-ADL score, MG-QOL15r score, and MG Composite score. In some embodiments, subjects may be monitored for MG characteristics over time. Such monitoring may be carried out over the course of MG disease. Monitoring may be carried out over the course of disease treatment. In some embodiments, subject evaluation or monitoring is carried out to assess changes in MG characteristics during or after subject treatment with zilucoplan.

In some embodiments, zilucoplan treated subjects are evaluated or monitored for QMG score. As described previously, the QMG is a standardized and validated quantitative strength scoring system that was developed specifically for MG and has been used previously in clinical trials. The scoring system assesses 13 items relating to ocular, bulbar, and limb function (Barnet, C. et al. 2015. J Neuromuscul Dis. 2:301-11). Each item is scored from 0-3. Maximum total score is 39. Higher scores are representative of more severe impairment. Recent data suggest that improvements in the QMG score of 2 to 3 points may be considered clinically meaningful, depending upon disease severity [Barohn R J et al. 1998. Ann N Y Acad Sci. 841:769-772; Katzberg H D et al. 2014. Muscle Nerve. 49(5):661-665, the contents of which are herein incorporated by reference in their entirety]. Subjects being assessed for QMG score may be pulled from MG therapies for at least 10 hours prior to QMG score assessment. The MG therapies may include acetylcholinesterase inhibitor therapy (e.g., pyridostigmine treatment) for at least 10 hours prior to QMG score assessment.

In some embodiments, change in QMG score may be a primary efficacy endpoint. Treated subject QMG score may be reduced. The QMG score may be reduced by at least 3 points. The QMG score may be reduced at or before 12 weeks of zilucoplan treatment. Treated subject QMG score may be monitored over the course of zilucoplan treatment.

In some embodiments, zilucoplan treated subject evaluations may include testing and/or monitoring for one or more of MG-ADL score, MG-QOL15r score, and MG Composite score. Such scores may be evaluated as secondary efficacy endpoints. As explained previously, The MG-ADL is a brief 8-item survey designed to evaluate MG symptom severity. Each item is scored from 0-3. Maximum total score is 24. Higher scores are associated with more severe symptoms of MG. The MG-ADL has been shown to correlate with other validated MG outcome measures (e.g., MG-QOL15r), and a 2-point improvement in MG-ADL score is considered clinically meaningful [Wolfe G I et al. 1999. Neurology. 52(7):1487-9; Muppidi S et al. 2011. Muscle Nerve. 44(5):727-31, the contents of which are herein incorporated by reference in their entirety]. The MG-QOL15r, as explained previously, is a 15-item survey that was designed to assess quality of life in patients with MG based on patient reporting. Each item is scored from 0-2. Maximum total score is 30. Higher scores indicate more severe impact of the disease on aspects of patient life [Burns, T M et al. 2010. Muscle Nerve. 41(2):219-26; Burns T M et al. 2016. Muscle Nerve. 54(6):1015-22, the contents of which are herein incorporated by reference in their entirety]. The MG Composite is a 10-item scale that has been used to measure the clinical status of patients with MG, both in the practice setting and in clinical trials, in order to evaluate treatment response (Burns, T. M. et al., 2008. Muscle Nerve. 38:1553-62). 10 items are assessed related to ocular, bulbar, respiratory, neck, and limb function. Items weighted, with scores ranging from 0-9. Maximum total score is 50. Higher scores in the MG Composite indicate more severe impairment due to the disease. A 3-point change in this instrument is considered clinically meaningful [Burns, T. M. et al. 2010. Neurology. 74(18): 1434-40; Sadjadi, D B et al. 2012. Neurology. 2016; 87(4):419-425, the contents of which are herein incorporated by reference in their entirety].

Testing or monitoring for MG-ADL, MG-QOL15r, and/or MG Composite score may be used to identify changes from baseline score. As used herein, the term “baseline score” refers to a score obtained before initial treatment. Baseline scores may be scores obtained between a switch from one treatment to another. The switch may be from a placebo to an active pharmaceutical compound. In some embodiments, zilucoplan treatment may be evaluated for reduction in MG-ADL score of at least 2 points. The reduction may occur at or before 12 weeks of zilucoplan treatment. In some embodiments, zilucoplan treatment may be evaluated for reduction in MG Composite score of at least 3 points. The reduction may occur at or before 12 weeks of zilucoplan treatment.

In some embodiments, zilucoplan treatment leads to reduced subject symptom expression. The reduced subject symptom expression may exceed reduced subject symptom expression associated with eculizumab administration.

### Evaluation Methods

In some embodiments, the present disclosure provides methods of evaluating treatments for MG. Such methods may include screening evaluation candidates for at least one evaluation participation criteria. As used herein, the term “evaluation candidate” refers to any individual being considered for participation in an evaluation (e.g., a clinical study). “Evaluation participation criteria” refers to a metric or factor used to select individuals to include in an evaluation. Evaluation candidates selected for participation in an evaluation are referred to herein as “evaluation participants.” In some embodiments, methods of evaluating treatments for MG may include screening an evaluation candidate for at least one evaluation participation criteria; selecting an evaluation participant: administering the treatment for MG to the evaluation participant, and assessing at least one efficacy endpoint.

In some embodiments, evaluation participation criteria include MG diagnosis. MG diagnosis may include gMG diagnosis. Diagnosis of gMG may be made according to MGFA criteria. In some embodiments, evaluation participation criteria include QMG score. Evaluation participant selections may require evaluation candidate QMG scores of ≥12. Some evaluation candidates may have received at least one alternative MG treatment (i.e., alternative to the treatment for MG being tested, such as standard of care treatments) prior to screening. In some embodiments, such candidates may be assessed for QMG score at least 10 hours after most recent alternative MG treatment. Alternative MG treatments may include standard of care MG treatments, including, but not limited to, cholinesterase inhibitor treatment, acetylcholinesterase inhibitor treatment, pyridostigmine treatment, corticosteroid treatment, and immunosuppressive drug treatment. Evaluation participant selection may require a score of ≥2 for ≥4 QMG test items.

In some embodiments, evaluation participation criteria include evaluation candidate age. In some embodiments, evaluation candidates must be between 18 and 85 years old.

Evaluation participation criteria may include candidate biomarker levels. In some embodiments, biomarkers include acetylcholinesterase receptor (AChR) autoantibody levels. AChR autoantibodies may lead to disease by binding AChR and stimulating complement activation. Accordingly, AChR autoantibody levels may be a good indicator of susceptibility to complement-mediated disease.

Evaluation participation criteria may include candidate prior and current alternative MG treatment status. In some embodiments, evaluation participants are selected based consistency of current or former alternative MG treatments. In some embodiments, candidates with no recent change in corticosteroid dose or immunosuppressive therapy are selected. Candidates with corticosteroid treatment dose or immunosuppressive therapy regimen changes within the past 30 days may be excluded from evaluation participation.

Evaluation participation criteria may include pregnancy status. In some embodiments, pregnant subjects may be excluded from evaluation participation. Pregnancy status screening may be carried out by serum pregnancy test. In some embodiments, pregnancy screening may include urine pregnancy testing.

Methods of evaluating treatments for MG may include administering treatments for MG to evaluation participants over an evaluation period. As used herein, the term “evaluation period” refers to a time frame over which a particular study takes place. Treatments may be administered over evaluation periods of from about one day to about 24 weeks. Some evaluation periods are about 12 weeks or longer. Evaluation participants may continue to receive standard of care gMG therapies over evaluation periods. Such therapies may include, but are not limited to, cholinesterase inhibitor treatment, acetylcholinesterase inhibitor treatment, pyridostigmine treatment, corticosteroid treatment, and/or immunosuppressive drug treatment.

Efficacy endpoints may include certain scores or changes in scores associated with assessments for individuals with MG. Such assessments may include, but are not limited to, QMG score, MG-ADL score, MG-QOL15r score, and MG Composite score. In some embodiments, efficacy endpoints include QMG score reduction. Efficacy endpoints may include at least 3 point reductions in QMG score. For evaluation participants receiving alternative MG treatments (e.g., acetylcholinesterase inhibitor treatment) during the evaluation period, one or more of those treatments may be withheld for at least 10 hours prior to QMG score assessment. In some embodiments, efficacy endpoints include reduction in one or more of MG-ADL score, MG-QOL15r score, and MG Composite score in relation to baseline score. Efficacy endpoints may include 2-point reduction in MG-ADL score over baseline score. The reduction in MG-ADL score may occur at or before 12 weeks of treatment for MG.

In some embodiments, assessing efficacy endpoints includes a set of assessments. The set of assessments may be carried out in a particular order. In some embodiments, the set of assessments are carried out in the order of: (1) assessing evaluation participant MG-QOL15r score; (2) assessing evaluation participant MG-ADL score: (3) assessing evaluation participant QMG score; and (4) assessing evaluation participant MG Composite score.

Assessments for efficacy endpoints may be carried out on one or more occasions after administering treatments for MG. Such assessments may be carried out at specific times and/or dates or may be carried out on a recurring basis (e.g., hourly, daily, weekly, monthly, or combinations thereof). In some embodiments, assessments are carried out 1 week, 2 weeks, 4 weeks, 8 weeks, and/or 12 weeks after starting administration of treatments for MG.

### Formulations

In some embodiments, compounds or compositions, e.g., pharmaceutical compositions, of the present disclosure are formulated in aqueous solutions. In some cases, aqueous solutions further include one or more salt and/or one or more buffering agent. Salts may include sodium chloride which may be included at concentrations of from about 0.05 mM to about 50 mM, from about 1 mM to about 100 mM, from about 20 mM to about 200 mM, or from about 50 mM to about 500 mM. Further solutions may include at least 500 mM sodium chloride. In some cases, aqueous solutions include sodium phosphate. Sodium phosphate may be included in aqueous solutions at a concentration of from about 0.005 mM to about 5 mM, from about 0.01 mM to about 10 mM, from about 0.1 mM to about 50 mM, from about 1 mM to about 100 mM, from about 5 mM to about 150 mM, or from about 10 mM to about 250 mM. In some cases, at least 250 mM sodium phosphate concentrations are used.

Compositions of the present disclosure may include C5 inhibitors at a concentration of from about 0.001 mg/mL to about 0.2 mg/mL, from about 0.01 mg/mL to about 2 mg/mL, from about 0.1 mg/mL to about 10 mg/mL, from about 0.5 mg/mL to about 5 mg/mL, from about 1 mg/mL to about 20 mg/mL, from about 15 mg/mL to about 40 mg/mL, from about 25 mg/mL to about 75 mg/mL, from about 50 mg/mL to about 200 mg/mL, or from about 100 mg/mL to about 400 mg/mL. In some cases, compositions include C5 inhibitors at a concentration of at least 400 mg/mL.

Compositions of the present disclosure may include C5 inhibitors at a concentration of approximately, about or exactly any of the following values: 0.001 mg/mL, 0.2 mg/mL, 0.01 mg/mL, 2 mg/mL, 0.1 mg/mL, 10 mg/mL, 0.5 mg/mL, 5 mg/mL, 1 mg/mL, 20 mg/mL, 15 mg/mL, 40 mg/mL, 25 mg/mL, 75 mg/mL, 50 mg/mL, 200 mg/mL, 100 mg/mL, or 400 mg/mL. In some cases, compositions include C5 inhibitors at a concentration of at least 40 mg/mL.

In some embodiments, compositions of the present disclosure include aqueous compositions including at least water and a C5 inhibitor (e.g., a cyclic C5 inhibitor polypeptide). Aqueous C5 inhibitor compositions may further include one or more salt and/or one or more buffering agent. In some cases, aqueous compositions include water, a cyclic C5 inhibitor polypeptide, a salt, and a buffering agent.

Aqueous C5 inhibitor formulations may have pH levels of from about 2.0 to about 3.0, from about 2.5 to about 3.5, from about 3.0 to about 4.0, from about 3.5 to about 4.5, from about 4.0 to about 5.0, from about 4.5 to about 5.5, from about 5.0 to about 6.0, from about 5.5 to about 6.5, from about 6.0 to about 7.0, from about 6.5 to about 7.5, from about 7.0 to about 8.0, from about 7.5 to about 8.5, from about 8.0 to about 9.0, from about 8.5 to about 9.5, or from about 9.0 to about 10.0.

In some cases, compounds and compositions of the present disclosure are prepared according to good manufacturing practice (GMP) and/or current GMP (cGMP). Guidelines used for implementing GMP and/or cGMP may be obtained from one or more of the US Food and Drug Administration (FDA), the World Health Organization (WHO), and the International Conference on Harmonization (ICH).

### Dosage and Administration

For treatment of human subjects, C5 inhibitors (e.g., zilucoplan and/or active metabolites or variants thereof) may be formulated as pharmaceutical compositions. Depending on the subject to be treated, the mode of administration, and the type of treatment desired (e.g., prevention, prophylaxis, or therapy) C5 inhibitors may be formulated in ways consonant with these parameters. A summary of such techniques is found in Remington: The Science and Practice of Pharmacy, 21st Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

C5 inhibitors (e.g., zilucoplan and/or active metabolites or variants thereof) may be provided in a therapeutically effective amount. In some cases, a therapeutically effective amount of a C5 inhibitor may be achieved by administration of a dose of from about 0.1 mg to about 1 mg, from about 0.5 mg to about 5 mg, from about 1 mg to about 20 mg, from about 5 mg to about 50 mg, from about 10 mg to about 100 mg, from about 20 mg to about 200 mg, or at least 200 mg of one or more C5 inhibitors.

In some embodiments, subjects may be administered a therapeutic amount of a C5 inhibitor (e.g., zilucoplan and/or active metabolites or variants thereof) based on the weight of such subjects. In some cases, C5 inhibitors are administered at a dose of from about 0.001 mg/kg to about 1.0 mg/kg, from about 0.01 mg/kg to about 2.0 mg/kg, from about 0.05 mg/kg to about 5.0 mg/kg, from about 0.03 mg/kg to about 3.0 mg/kg, from about 0.01 mg/kg to about 10 mg/kg, from about 0.1 mg/kg to about 2.0 mg/kg, from about 0.2 mg/kg to about 3.0 mg/kg, from about 0.4 mg/kg to about 4.0 mg/kg, from about 1.0 mg/kg to about 5.0 mg/kg, from about 2.0 mg/kg to about 4.0 mg/kg, from about 1.5 mg/kg to about 7.5 mg/kg, from about 5.0 mg/kg to about 15 mg/kg, from about 7.5 mg/kg to about 12.5 mg/kg, from about 10 mg/kg to about 20 mg/kg, from about 15 mg/kg to about 30 mg/kg, from about 20 mg/kg to about 40 mg/kg, from about 30 mg/kg to about 60 mg/kg, from about 40 mg/kg to about 80 mg/kg, from about 50 mg/kg to about 100 mg/kg, or at least 100 mg/kg. Such ranges may include ranges suitable for administration to human subjects. Dosage levels may be highly dependent on the nature of the condition; drug efficacy: the condition of the patient; the judgment of the practitioner; and the frequency and mode of administration. In some embodiments, zilucoplan and/or active metabolites or variants thereof may be administered at a dose of from about 0.01 mg/kg to about 10 mg/kg. In some cases, zilucoplan and/or active metabolites or variants thereof may be administered at a dose of from about 0.1 mg/kg to about 3 mg/kg.

In some cases, C5 inhibitors (e.g., zilucoplan and/or active metabolites or variants thereof) are provided at concentrations adjusted to achieve a desired level of the C5 inhibitor in a sample, biological system, or subject (e.g., plasma level in a subject). In some cases, desired concentrations of C5 inhibitors in a sample, biological system, or subject may include concentrations of from about 0.001 μM to about 0.01 μM, from about 0.005 μM to about 0.05 μM, from about 0.02 μM to about 0.2 μM, from about 0.03 μM to about 0.3 μM, from about 0.05 μM to about 0.5 μM, from about 0.01 μM to about 2.0 μM, from about 0.1 μM to about 50 μM, from about 0.1 μM to about 10 μM, from about 0.1 μM to about 5 μM, from about 0.2 μM to about 20 μM, from about 5 μM to about 100 μM, or from about 15 μM to about 200 μM. In some cases, desired concentrations of C5 inhibitors in subject plasma may be from about 0.1 μg/mL to about 1000 μg/mL. The desired concentration of C5 inhibitors in subject plasma may be from about 0.01 μg/mL to about 2 μg/mL, from about 0.02 μg/mL to about 4 μg/mL, from about 0.05 μg/mL to about 5 μg/mL, from about 0.1 μg/mL to about 1.0 μg/mL, from about 0.2 μg/mL to about 2.0 μg/mL, from about 0.5 μg/mL to about 5 μg/mL, from about 1 μg/mL to about 5 μg/mL, from about 2 μg/mL to about 10 μg/mL, from about 3 μg/mL to about 9 μg/mL, from about 5 μg/mL to about 20 μg/mL, from about 10 μg/mL to about 40 μg/mL, from about 30 μg/mL to about 60 μg/mL, from about 40 μg/mL to about 80 μg/mL, from about 50 μg/mL to about 100 μg/mL, from about 75 μg/mL to about 150 μg/mL, or at least 150 μg/mL. In other embodiments, C5 inhibitors are administered at a dose sufficient to achieve a maximum serum concentration (Cₘₐₓ) of at least 0.1 μg/mL, at least 0.5 μg/mL, at least 1 μg/mL, at least 5 μg/mL, at least 10 μg/mL, at least 50 μg/mL, at least 100 μg/mL, or at least 1000 μg/mL.

In some embodiments, C5 inhibitors (e.g., zilucoplan and/or active metabolites or variants thereof) are administered daily at a dose sufficient to deliver from about 0.1 mg/day to about 60 mg/day per kg weight of a subject. In some cases, the Cₘₐₓ achieved with each dose is from about 0.1 μg/mL to about 1000 μg/mL. In such cases, the area under the curve (AUC) between doses may be from about 200 μg*hr/mL to about 10,000 μg*hr/mL.

According to some methods of the present disclosure, C5 inhibitors (e.g., zilucoplan and/or active metabolites or variants thereof) are provided at concentrations needed to achieve a desired effect. In some cases, compounds and compositions of the disclosure are provided at an amount necessary to reduce a given reaction or process by half. The concentration needed to achieve such a reduction is referred to herein as the half maximal inhibitory concentration, or “IC₅₀.” Alternatively, compounds and compositions of the disclosure may be provided at an amount necessary to increase a given reaction, activity or process by half. The concentration needed for such an increase is referred to herein as the half maximal effective concentration or “EC₅₀.”

C5 inhibitors (e.g., zilucoplan and/or active metabolites or variants thereof) may be present in amounts totaling 0.1-95% by weight of the total weight of the composition. In some cases, C5 inhibitors are provided by intravenous (IV) administration. In some cases, C5 inhibitors are provided by subcutaneous (SC) administration.

SC administration of C5 inhibitors (e.g., zilucoplan and/or active metabolites or variants thereof) may, in some cases, provide advantages over IV administration. SC administration may include self-administration by using an administration device, such as a self-administration device. As used herein, the term “self-administration” refers to any form of therapeutic delivery that is carried out wholly or in part by the recipient of a therapeutic treatment. Self-administration devices may include self-injection devices. Self-administration treatment may be advantageous in that patients can provide treatment to themselves in their own home, avoiding the need to travel to a provider or medical facility. Further, SC treatment may allow patients to avoid long-term complications associated with IV administration, such as infections, loss of venous access, local thrombosis, and hematomas. In some embodiments, self-administration using a self-injection device may increase patient compliance, patient satisfaction, quality of life, reduce treatment costs and/or drug requirements.

In some cases, daily SC administration provides steady-state C5 inhibitor concentrations that are reached within 1-3 doses, 2-3 doses, 3-5 doses, or 5-10 doses. In some cases, daily SC doses of from about 0.1 mg/kg to about 0.3 mg/kg may achieve sustained C5 inhibitor levels greater than or equal to 2.5 μg/mL and/or inhibition of complement activity of greater than 90%.

C5 inhibitors (e.g., zilucoplan and/or active metabolites or variants thereof) may exhibit slow absorption kinetics (time to maximum observed concentration of greater than 4-8 hours) and high bioavailability (from about 75% to about 100%) after SC administration.

In some embodiments, dosage and/or administration are altered to modulate the half-life (t_{1/2}) of C5 inhibitor levels in a subject or in subject fluids (e.g., plasma). In some cases, t_{1/2} is at least 1 hour, at least 2 hrs, at least 4 hrs, at least 6 hrs, at least 8 hrs, at least 10 hrs, at least 12 hrs, at least 16 hrs, at least 20 hrs, at least 24 hrs, at least 36 hrs, at least 48 hrs, at least 60 hrs, at least 72 hrs, at least 96 hrs, at least 5 days, at least 6 days, at least 7 days, at least 8 days, at least 9 days, at least 10 days, at least 11 days, at least 12 days, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, or at least 16 weeks.

In some embodiments, C5 inhibitors (e.g., zilucoplan and/or active metabolites or variants thereof) may exhibit long terminal t_{1/2}. Extended terminal t_{1/2} may be due to extensive target binding and/or additional plasma protein binding. In some cases, C5 inhibitors exhibit t_{1/2} values greater than 24 hours in both plasma and whole blood. In some cases, C5 inhibitors do not lose functional activity after incubation in human whole blood at 37° C. for 16 hours.

In some embodiments, dosage and/or administration are altered to modulate the steady state volume of distribution of C5 inhibitors. In some cases, the steady state volume of distribution of C5 inhibitors is from about 0.1 mL/kg to about 1 mL/kg, from about 0.5 mL/kg to about 5 mL/kg, from about 1 mL/kg to about 10 mL/kg, from about 5 mL/kg to about 20 mL/kg, from about 15 mL/kg to about 30 mL/kg, from about 10 mL/kg to about 200 mL/kg, from about 20 mL/kg to about 60 mL/kg, from about 30 mL/kg to about 70 mL/kg, from about 50 mL/kg to about 200 mL/kg, from about 100 mL/kg to about 500 mL/kg, or at least 500 mL/kg. In some cases, the dosage and/or administration of C5 inhibitors is adjusted to ensure that the steady state volume of distribution is equal to at least 50% of total blood volume. In some embodiments, C5 inhibitor distribution may be restricted to the plasma compartment.

In some embodiments, C5 inhibitors (e.g., zilucoplan and/or active metabolites or variants thereof) exhibit a total clearance rate of from about 0.001 mL/hr/kg to about 0.01 mL/hr/kg, from about 0.005 mL/hr/kg to about 0.05 mL/hr/kg, from about 0.01 mL/hr/kg to about 0.1 mL/hr/kg, from about 0.05 mL/hr/kg to about 0.5 mL/hr/kg, from about 0.1 mL/hr/kg to about 1 mL/hr/kg, from about 0.5 mL/hr/kg to about 5 mL/hr/kg, from about 0.04 mL/hr/kg to about 4 mL/hr/kg, from about 1 mL/hr/kg to about 10 mL/hr/kg, from about 5 mL/hr/kg to about 20 mL/hr/kg, from about 15 mL/hr/kg to about 30 mL/hr/kg, or at least 30 mL/hr/kg.

Time periods for which maximum concentration of C5 inhibitors in subjects (e.g., in subject serum) are maintained (Tₘₐₓ values) may be adjusted by altering dosage and/or administration (e.g., subcutaneous administration). In some cases, C5 inhibitors have Tₘₐₓ values of from about 1 min to about 10 min. from about 5 min to about 20 min, from about 15 min to about 45 min, from about 30 min to about 60 min, from about 45 min to about 90 min, from about 1 hour to about 48 hrs, from about 2 hrs to about 10 hrs, from about 5 hrs to about 20 hrs, from about 10 hrs to about 60 hrs, from about 1 day to about 4 days, from about 2 days to about 10 days, or at least 10 days.

In some embodiments, C5 inhibitors (e.g., zilucoplan and/or active metabolites or variants thereof) may be administered without off-target effects. In some cases, C5 inhibitors do not inhibit hERG (human ether-a-go-go related gene), even with concentrations less than or equal to 300 μM. SC injection of C5 inhibitors with dose levels up to 10 mg/kg may be well tolerated and not result in any adverse effects of the cardiovascular system (e.g., elevated risk of prolonged ventricular repolarization) and/or respiratory system.

C5 inhibitor doses may be determined using the no observed adverse effect level (NOAEL) observed in another species. Such species may include, but are not limited to monkeys, rats, rabbits, and mice. In some cases, human equivalent doses (HEDs) may be determined by allometric scaling from NOAELs observed in other species. In some cases, HEDs result in therapeutic margins of from about 2-fold to about 5-fold, from about 4-fold to about 12-fold, from about 5-fold to about 15-fold, from about 10-fold to about 30-fold, or at least 30-fold. In some cases, therapeutic margins are determined by using exposure in primates and estimated human Cₘₐₓ levels in humans.

In some embodiments, C5 inhibitors of the present disclosure allow for a rapid washout period in cases of infection where prolonged inhibition of the complement system prove detrimental.

C5 inhibitor administration according to the present disclosure may be modified to reduce potential clinical risks to subjects. Infection with Neisseria meningitidis is a known risk of C5 inhibitors, including eculizumab. In some cases, risk of infection with Neisseria meningitides is minimized by instituting one or more prophylactic steps. Such steps may include the exclusion of subjects who may already be colonized by these bacteria. In some cases, prophylactic steps may include coadministration with one or more antibiotics. In some cases, ciprofloxacin may be co-administered. In some cases, ciprofloxacin may be co-administered orally at a dose of from about 100 mg to about 1000 mg (e.g., 500 mg).

In some embodiments, C5 inhibitors (e.g., zilucoplan and/or active metabolites or variants thereof) are administered at a frequency of every hour, every 2 hrs, every 4 hrs, every 6 hrs, every 12 hrs, every 18 hrs, every 24 hrs, every 36 hrs, every 72 hrs, every 84 hrs, every 96 hrs, every 5 days, every 7 days, every 10 days, every 14 days, every week, every two weeks, every 3 weeks, every 4 weeks, every month, every 2 months, every 3 months, every 4 months, every 5 months, every 6 months, every year, or at least every year. In some cases, C5 inhibitors are administered once daily or administered as two, three, or more sub-doses at appropriate intervals throughout the day.

In some embodiments, C5 inhibitors are administered in multiple daily doses. In some cases, C5 inhibitors are administered daily for 7 days. In some cases, C5 inhibitors are administered daily for 7 to 100 days. In some cases, C5 inhibitors are administered daily for at least 100 days. In some cases, C5 inhibitors are administered daily for an indefinite period.

Methods of the present disclosure may include administering a C5 inhibitor (e.g., zilucoplan and/or active metabolites or variants thereof) at a daily dose of from about 0.1 mg/kg to about 0.3 mg/kg. In some embodiments, a C5 inhibitor (e.g., zilucoplan and/or active metabolites or variants thereof) is administered at a daily dose of 0.3 mg/kg. Subject QMG score and/or MG-ADL score may be reduced as a result of administration. QMG score may be reduced by ≥3 points by 8 weeks of treatment. MG-ADL score may be reduced by ≥2 points by 8 weeks of treatment. Risk of need for rescue therapy (IVIG or plasma exchange) may be reduced.

C5 inhibitors delivered intravenously may be delivered by infusion over a period of time, such as over a 5 minute, 10 minute, 15 minute, 20 minute, or 25 minute period. The administration may be repeated, for example, on a regular basis, such as hourly, daily, weekly, biweekly (i.e., every two weeks), for one month, two months, three months, four months, or more than four months. After an initial treatment regimen, treatments may be administered on a less frequent basis. For example, after biweekly administration for three months, administration may be repeated once per month, for six months or a year or longer. C5 inhibitor administration may reduce, lower, increase or alter binding or any physiologically deleterious process (e.g., in a cell, tissue, blood, urine or other compartment of a patient) by at least 10%, at least 15%, at least 20%, at least 25%, at least 30%, at least 40%, at least 50%, at least 60%, at least 70%, at least 80% or at least 90% or more.

Before administration of a full dose of C5 inhibitor and/or C5 inhibitor composition, patients can be administered a smaller dose, such as 5% of a full dose, and monitored for adverse effects, such as an allergic reaction or infusion reaction, or for elevated lipid levels or blood pressure. In another example, patients can be monitored for unwanted immunostimulatory effects, such as increased cytokine (e.g., TNF-alpha, IL-1, IL-6, or IL-10) levels.

Genetic predisposition plays a role in the development of some diseases or disorders. Therefore, patients in need of C5 inhibitors may be identified by family history analysis, or, for example, screening for one or more genetic markers or variants. Healthcare providers (e.g., doctors or nurses) or family members may analyze family history information before prescribing or administering therapeutic compositions of the present disclosure.

### III. Kits and Devices

In some embodiments, the present disclosure provides kits and devices. Such kits and devices may include any of the compounds or compositions described herein. In a non-limiting example, zilucoplan may be included.

Devices of the present disclosure may include administration devices. As used herein, the term “administration device” refers to any tool for providing a substance to a recipient. Administration devices may include self-administration devices. As used herein, the term “self-administration device” refers to any tool used for providing a substance to a recipient, wherein use of the tool is carried out wholly or in part by the recipient. Self-administration devices may include self-injection devices. “Self-injection devices” are self-administration devices that enable individuals to subcutaneously administer substances to their own body. Self-injection devices may include prefilled syringes. As used herein, the term “prefilled syringe” refers to a syringe that has been loaded with a substance or cargo prior to access or use by an operator of the syringe. For example, prefilled syringes (also referred to herein as “pre-loaded syringes”) may be filled with a therapeutic composition prior to packaging in a kit; prior to syringe shipment to a distributor, administrator, or operator; or prior to access by a subject using the syringe for self-administration. Due to cyclic peptide stability, cyclic peptide inhibitors (e.g., zilucoplan) are especially well suited for manufacture, storage, and distribution in pre-loaded syringes. Further, pre-loaded syringes are especially well suited for self-administration (i.e., administration by a subject, without the aid of a medical professional). Self-administration represents a convenient way for subjects to obtain treatments without relying on medical professionals who may be located at a distance or are otherwise difficult to access. This makes self-administration options well suited for treatments requiring frequent injections (e.g., daily injections).

Prefilled syringes may be of any material (e.g., glass, plastic, or metal). In some embodiments, prefilled syringes are glass syringes. Prefilled syringes may include maximum fill volumes (meaning the largest amount of liquid that can be contained) of at least 0.1 ml, at least 0.2 ml, at least 0.3 ml, at least 0.4 ml, at least 0.5 ml, at least 0.75 ml, at least 1.0 ml, at least 1.5 ml, at least 2.0 ml, at least 5.0 ml, at least 10 ml, or more than 10 ml. Syringes may include needles. The needles may be of any gauge. In some embodiments, syringes include 29-gauge needles. The needles may be assembled with syringes or attached prior to syringe use. Self-injection devices may include BD ULTRASAFE PLUS™ self-administration devices (BD, Franklin Lakes, N.J.).

Administration devices may include self-injection devices that include a syringe and needle and a predetermined volume of a zilucoplan composition. The zilucoplan composition may be a pharmaceutical composition. The composition may include a zilucoplan concentration of from about 1 mg/mL to about 200 mg/mL. In some embodiments, the zilucoplan concentration is about 40 mg/mL. Predetermined volumes may be predetermined based on subject body weight. In some embodiments, predetermined zilucoplan composition volumes are modified to facilitate zilucoplan administration to a subject at a dose of from about 0.1 mg/kg to about 0.6 mg/kg. Volumes may be modified to facilitate 0.3 mg/kg zilucoplan dosing. The self-injection device may include a BD ULTRASAFE PLUS™ self-administration device. In some embodiments, administration devices are prepared for storage at specific temperatures or temperature ranges. Some administration devices may be prepared for storage at room temperature. Some administration devices may be prepared for storage between from about 2° C. to about 8° C.

Pre-filled syringes may include ULTRASAFE PLUS™ passive needle guards (Becton Dickenson, Franklin Lakes, N.J.). Other pre-filled syringes may include injection pens. Injection pens may be multi-dose pens. Some pre-filled syringes may include a needle. In some embodiments, the needle gauge is from about 20 to about 34. The needle gauge may be from about 29 to about 31.

In some embodiments, kits of the present disclosure include kits carrying out methods of treating MG described herein. Such kits may include one or more administration devices described herein and instructions for kit usage.

Kit components may be packaged in liquid (e.g., aqueous or organic) media or in dry (e.g., lyophilized) form. Kits may include containers that may include, but are not limited to vials, test tubes, flasks, bottles, syringes, or bags. Kit containers may be used to aliquot, store, preserve, insulate, and/or protect kit components. Kit components may be packaged together or separately. Some kits may include containers of sterile, pharmaceutically acceptable buffer and/or other diluent (e.g., phosphate buffered saline). In some embodiments, kits include containers of kit components in dry form with separate containers of solution for dissolving dried components. In some embodiments, kits include a syringe for administering one or more kit components.

When polypeptides are provided as a dried powder it is contemplated that between 10 micrograms and 1000 milligrams of polypeptide, or at least or at most those amounts are provided in kits.

Containers may include at least one vial, test tube, flask, bottle, syringe and/or other receptacle, into which polypeptide formulations may be placed, preferably, suitably allocated. Kits may also include containers for sterile, pharmaceutically acceptable buffer and/or other diluent.

Kits may include instructions for employing kit components as well the use of any other reagent not included in the kit. Instructions may include variations that can be implemented.

Kits may include one or more items for addressing syringe wounds. Such items may include, but are not limited to, alcohol wipes and wound dressings (e.g., cotton balls, mesh pads, bandages, tape, gauze, etc.). Kits may further include disposal containers for disposal of used kit components. Disposal containers may be designed for disposal of sharp objects, such as needles and syringes. Some kits may include instructions for sharp object disposal.

In some embodiments, kits of the present disclosure include zilucoplan in powdered form or in solution (e.g., as pharmaceutical compositions). Solutions may be aqueous solutions. Solutions may include PBS. Zilucoplan solutions may include from about 4 mg/ml to about 200 mg/ml zilucoplan. In some embodiments, zilucoplan solutions include about 40 mg/ml zilucoplan. Zilucoplan solutions may include preservatives. In some embodiments, zilucoplan solutions are preservative-free.

In some embodiments, kits are prepared for storage at specific temperatures or temperature ranges. Some kits may be prepared for storage at room temperature. Some kits may be prepared for storage between from about 2° C. to about 8° C.

### IV. Definitions

Bioavailability: As used herein, the term “bioavailability” refers to the systemic availability of a given amount of a compound (e.g., C5 inhibitor) administered to a subject. Bioavailability can be assessed by measuring the area under the curve (AUC) or the maximum serum or plasma concentration (Cₘₐₓ) of the unchanged form of a compound following administration of the compound to a subject. AUC is a determination of the area under the curve when plotting the serum or plasma concentration of a compound along the ordinate (Y-axis) against time along the abscissa (X-axis). Generally, the AUC for a particular compound can be calculated using methods known to those of ordinary skill in the art and/or as described in G. S. Banker, Modern Pharmaceutics, Drugs and the Pharmaceutical Sciences, v. 72, Marcel Dekker, New York, Inc., 1996, the contents of which are herein incorporated by reference in their entirety.

Biological system: As used herein, the term “biological system” refers to a cell, a group of cells, a tissue, an organ, a group of organs, an organelle, a biological fluid, a biological signaling pathway (e.g., a receptor-activated signaling pathway, a charge-activated signaling pathway, a metabolic pathway, a cellular signaling pathway, etc.), a group of proteins, a group of nucleic acids, or a group of molecules (including, but not limited to biomolecules) that carry out at least one biological function or biological task within cellular membranes, cellular compartments, cells, cell cultures, tissues, organs, organ systems, organisms, multicellular organisms, biological fluids, or any biological entities. In some embodiments, biological systems are cell signaling pathways that include intracellular and/or extracellular signaling biomolecules. In some embodiments, biological systems include proteolytic cascades (e.g., the complement cascade).

Buffering agent: As used herein, the term “buffering agent” refers to a compound used in a solution for the purposes of resisting changes in pH. Such compounds may include, but are not limited to acetic acid, adipic acid, sodium acetate, benzoic acid, citric acid, sodium benzoate, maleic acid, sodium phosphate, tartaric acid, lactic acid, potassium metaphosphate, glycine, sodium bicarbonate, potassium phosphate, sodium citrate, and sodium tartrate.

Clearance rate: As used herein, the term “clearance rate” refers to the velocity at which a particular compound is cleared from a biological system or fluid.

Compound: As used herein, the term “compound,” refers to a distinct chemical entity. In some embodiments, a particular compound may exist in one or more isomeric or isotopic forms (including, but not limited to stereoisomers, geometric isomers and isotopes). In some embodiments, a compound is provided or utilized in only a single such form. In some embodiments, a compound is provided or utilized as a mixture of two or more such forms (including, but not limited to a racemic mixture of stereoisomers). Those of skill in the art will appreciate that some compounds exist in different forms, show different properties and/or activities (including, but not limited to biological activities). In such cases it is within the ordinary skill of those in the art to select or avoid particular forms of a compound for use in accordance with the present disclosure. For example, compounds that contain asymmetrically substituted carbon atoms can be isolated in optically active or racemic forms.

Cyclic or Cyclized: As used herein, the term “cyclic” refers to the presence of a continuous loop. Cyclic molecules need not be circular, only joined to form an unbroken chain of subunits. Cyclic polypeptides may include a “cyclic loop,” formed when two amino acids are connected by a bridging moiety. The cyclic loop comprises the amino acids along the polypeptide present between the bridged amino acids. Cyclic loops may include 2, 3, 4, 5, 6, 7, 8, 9, 10 or more amino acids.

Downstream event: As used herein, the term “downstream” or “downstream event,” refers to any event occurring after and/or as a result of another event. In some cases, downstream events are events occurring after and as a result of C5 cleavage and/or complement activation. Such events may include, but are not limited to, generation of C5 cleavage products, activation of MAC, hemolysis, and hemolysis-related disease (e.g., PNH).

Equilibrium dissociation constant: As used herein, the term “equilibrium dissociation constant” or “K_{D}” refers to a value representing the tendency of two or more agents (e.g., two proteins) to reversibly separate. In some cases, K_{D} indicates a concentration of a primary agent at which half of the total levels of a secondary agent are associated with the primary agent.

Half-life: As used herein, the term “half-life” or “t_{1/2}” refers to the time it takes for a given process or compound concentration to reach half of a final value. The “terminal half-life” or “terminal t_{1/2}” refers to the time needed for the plasma concentration of a factor to be reduced by half after the concentration of the factor has reached a pseudo-equilibrium.

Identity: As used herein, the term “identity” when referring to polypeptides or nucleic acids, refers to a comparative relationship between sequences. The term is used to describe the degree of sequence relatedness between polymeric sequences and may include the percentage of matching monomeric components with gap alignments (if any) addressed by a particular mathematical model or computer program (i.e., “algorithms”). Identity of related polypeptides can be readily calculated by known methods. Such methods include, but are not limited to, those described previously by others (Lesk, A. M., ed., Computational Molecular Biology, Oxford University Press, New York, 1988; Smith, D. W., ed., Biocomputing: Informatics and Genome Projects, Academic Press, New York, 1993: Griffin, A. M. et al., ed., Computer Analysis of Sequence Data, Part 1, Humana Press, New Jersey, 1994; von Heinje, G., Sequence Analysis in Molecular Biology, Academic Press, 1987; Gribskov, M. et al., ed., Sequence Analysis Primer, M. Stockton Press, New York, 1991; and Carillo et al., Applied Math, SIAM J, 1988, 48, 1073).

Inhibitor: As used herein, the term “inhibitor” refers to any agent that blocks or causes a reduction in the occurrence of a specific event; cellular signal; chemical pathway; enzymatic reaction: cellular process; interaction between two or more entities; biological event; disease: disorder: or condition.

Initial loading dose: As used herein, an “initial loading dose” refers to a first dose of a therapeutic agent that may differ from one or more subsequent doses. Initial loading doses may be used to achieve an initial concentration of a therapeutic agent or level of activity before subsequent doses are administered.

Intravenous: As used herein, the term “intravenous” refers to the area within a blood vessel. Intravenous administration typically refers to delivery of a compound into the blood through injection in a blood vessel (e.g., vein).

In vitro: As used herein, the term “in vitro” refers to events that occur in an artificial environment (e.g., in a test tube or reaction vessel, in cell culture, in a Petri dish, etc.), rather than within an organism (e.g., animal, plant, or microbe).

In vivo: As used herein, the term “in vivo” refers to events that occur within an organism (e.g., animal, plant, or microbe or cell or tissue thereof).

Lactam bridge: As used herein, the term “lactam bridge” refers to an amide bond that forms a bridge between chemical groups in a molecule. In some cases, lactam bridges are formed between amino acids in a polypeptide.

Linker: The term “linker” as used herein refers to a group of atoms (e.g., 10-1,000 atoms), molecule(s), or other compounds used to join two or more entities. Linkers may join such entities through covalent or non-covalent (e.g., ionic or hydrophobic) interactions. Linkers may include chains of two or more polyethylene glycol (PEG) units. In some cases, linkers may be cleavable.

Minute volume: As used herein, the term “minute volume” refers to the volume of air inhaled or exhaled from a subject's lungs per minute.

Non-proteinogenic: As used herein, the term “non-proteinogenic” refers to any non-natural proteins, such as those with non-natural components, such as non-natural amino acids.

Patient: As used herein, “patient” refers to a subject who may seek or be in need of treatment, requires treatment, is receiving treatment, will receive treatment, or a subject who is under the care of a trained professional for a particular disease or condition.

Pharmaceutical composition: As used herein, the term “pharmaceutical composition” refers to a composition with at least one active ingredient (e.g., a C5 inhibitor) in a form and amount that permits the active ingredient to be therapeutically effective.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers to any ingredient other than active agents (e.g., active agent zilucoplan and/or active metabolites thereof or variants thereof) present in a pharmaceutical composition and having the properties of being substantially nontoxic and non-inflammatory in a patient. In some embodiments, a pharmaceutically acceptable excipient is a vehicle capable of suspending or dissolving the active agent. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Plasma compartment: As used herein, the term “plasma compartment” refers to intravascular space occupied by blood plasma.

Salt: As used herein, the term “salt” refers to a compound made up of a cation with a bound anion. Such compounds may include sodium chloride (NaCl) or other classes of salts including, but not limited to acetates, chlorides, carbonates, cyanides, nitrites, nitrates, sulfates, and phosphates.

Sample: As used herein, the term “sample” refers to an aliquot or portion taken from a source and/or provided for analysis or processing. In some embodiments, a sample is from a biological source such as a tissue, cell or component part (e.g., a body fluid, including but not limited to blood, mucus, lymphatic fluid, synovial fluid, cerebrospinal fluid, saliva, amniotic fluid, amniotic cord blood, urine, vaginal fluid and semen). In some embodiments, a sample may be or include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, or organs. In some embodiments, a sample is or includes a medium, such as a nutrient broth or gel, which may contain cellular components, such as proteins. In some embodiments, a “primary” sample is an aliquot of the source. In some embodiments, a primary sample is subjected to one or more processing (e.g., separation, purification, etc.) steps to prepare a sample for analysis or other use.

Subcutaneous: As used herein, the term “subcutaneous” refers to the space underneath the skin. Subcutaneous administration is delivery of a compound beneath the skin.

Subject: As used herein, the term “subject” refers to any organism to which a compound or method in accordance with the disclosure may be administered or applied, e.g., for experimental, diagnostic, prophylactic, and/or therapeutic purposes. Typical subjects include animals (e.g., mammals such as mice, rats, rabbits, porcine subjects, non-human primates, and humans). In some applications, the subject is human.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutically effective amount: As used herein, the term “therapeutically effective amount” means an amount of an agent to be delivered (e.g., C5 inhibitor) that is sufficient, when administered to a subject suffering from or susceptible to a disease, disorder, and/or condition, to treat, improve symptoms of, diagnose, prevent, and/or delay the onset of the disease, disorder, and/or condition.

Tidal volume: As used herein, the term “tidal volume” refers to the normal lung volume of air displaced between breaths (in the absence of any extra effort).

Tₘₐₓ: As used herein, the term “Tₘₐₓ” refers to the time period for which maximum concentration of a compound in a subject or fluid is maintained.

Treating: As used herein, the term “treating” refers to partially or completely alleviating, ameliorating, improving, relieving, delaying onset of, inhibiting progression of, reducing severity of, and/or reducing incidence of one or more symptoms or features of a particular disease, disorder, and/or condition. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Treatment dose: As used herein. “treatment dose” refers to one or more doses of a therapeutic agent administered in the course of addressing or alleviating a therapeutic indication. Treatment doses may be adjusted to maintain a desired concentration or level of activity of a therapeutic agent in a body fluid or biological system.

Volume of distribution: As used herein, the term “volume of distribution” or “V_{dist}” refers to a fluid volume required to contain the total amount of a compound in the body at the same concentration as in the blood or plasma. The volume of distribution may reflect the extent to which a compound is present in the extravascular tissue. A large volume of distribution reflects the tendency of a compound to bind to tissue components compared with plasma protein components. In a clinical setting, V_{dist} can be used to determine a loading dose of a compound to achieve a steady state concentration of that compound.

### V. Equivalents and Scope

While various embodiments of the invention have been particularly shown and described, it will be understood by those skilled in the art that various changes in form and details may be made therein without departing from the spirit and scope of the invention as defined by the appended claims.

Those skilled in the art will recognize or be able to ascertain using no more than routine experimentation, many equivalents to the specific embodiments in accordance with the invention described herein. The scope of the present invention is not intended to be limited to the above description, but rather is as set forth in the appended claims.

In the claims, articles such as “a.” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the terms “consisting of” and “or including” are thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production: any method of use: etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Example 1. Preparation of Zilucoplan Aqueous Solution

Polypeptides were synthesized using standard solid-phase Fmoc/tBu methods. The synthesis was performed on a Liberty automated microwave peptide synthesizer (CEM, Matthews N.C.) using standard protocols with Rink amide resin, although other automated synthesizers without microwave capability may also be used. All amino acids were obtained from commercial sources. The coupling reagent used was 2-(6-chloro-1-H-benzotriazole-1yl)-1,1,3,3,-tetramethylaminium hexafluorophosphate (HCTU) and the base was diisopropylethylamine (DIEA). Polypeptides were cleaved from resin with 95% TFA, 2.5% TIS and 2.5% water for 3 hours and isolated by precipitation with ether. The crude polypeptides were purified on a reverse phase preparative HPLC using a C18 column, with an acetonitrile/water 0.1% TFA gradient from 20%-50% over 30 min. Fractions containing pure polypeptides were collected and lyophilized and all polypeptides were analyzed by LC-MS.

Zilucoplan (SEQ ID NO: 1; CAS Number: 1841136-73-9) was prepared as a cyclic peptide containing 15 amino acids (4 of which are non-natural amino acids), an acetylated N-terminus, and a C-terminal carboxylic acid. The C-terminal lysine of the core peptide has a modified side chain, forming a N-ε-(PEG24-γ-glutamic acid-N-α-hexadecanoyl) lysine reside. This modified side chain includes a polyethyleneglycol spacer (PEG24) attached to an L-γ glutamic acid residue that is derivatized with a palmitoyl group. The cyclization of zilucoplan is via a lactam bridge between the side-chains of L-Lys1 and L-Asp6. All of the amino acids in zilucoplan are L-amino acids. Zilucoplan has a molecular weight of 3562.23 g/mol and a chemical formula of C₁₇₂H₂₇₈N₂₄O₅₅.

Like eculizumab, zilucoplan blocks the proteolytic cleavage of C5 into C5a and C5b. Unlike eculizumab, zilucoplan can also bind to C5b and block C6 binding which prevents the subsequent assembly of the MAC.

Zilucoplan was prepared as an aqueous solution for injection containing 40 mg/mL of zilucoplan in a sterile, preservative-free formulation of 50 mM sodium phosphate and 76 mM sodium chloride at a pH of 7.0. The resulting composition was used to prepare a medicinal product, in accordance with current Good Manufacturing Practices (cGMPs), the medicinal product including a pre-filled 1 ml glass syringe with a 29 gauge, ½ inch staked needle placed within a BD ULTRASAFE PLUS™ (BD, Franklin Lakes, N.J.) self-administration device.

### Example 2. Zilucoplan Administration and Storage

Zilucoplan is administered by subcutaneous (SC) or intravenous (IV) injection and the dose administered (dose volume) is adjusted based on subject weight on a mg/kg basis. This is achieved using a set of fixed doses aligned to a set of weight brackets. In total, human dosing supports a broad weight range of 43 to 109 kg. Subjects who present with a higher body weight (>109 kg) are accommodated on a case-by-case basis, in consultation with a medical monitor.

Zilucoplan is stored at 2° C. to 8° C. [36° F. to 46° F.]. Once dispensed to subjects, zilucoplan is stored at controlled room temperature (20° C. to 25° C. [68° F. to 77° F.]) for up to 30 days and is protected from sources of excessive temperature fluctuations such as high heat or exposure to light. Storage of zilucoplan outside of room temperatures is preferably avoided. Zilucoplan may be stored for up to 30 days under these conditions.

### Example 3. Zilucoplan Myasthenia Gravis Treatment Evaluation

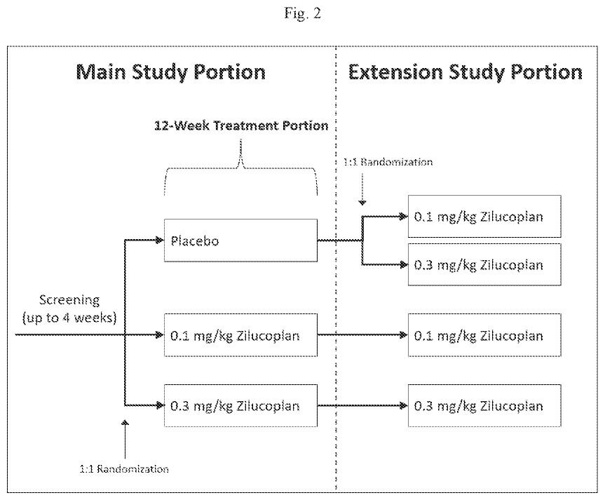

A multicenter, randomized, double-blind, placebo-controlled study was carried out to evaluate zilucoplan safety, tolerability, and preliminary efficacy in treating subjects with gMG. A schematic of the study design is presented in FIG. 2. During the study, subjects were randomized in a 1:1:1 ratio to receive daily SC doses of 0.1 mg/kg zilucoplan, 0.3 mg/kg zilucoplan, or matching placebo. Randomization was stratified based on screening Quantitative Myasthenia Gravis (QMG) score (≤17 versus ≥18).

The Main Portion of the study included a Screening Period of up to 4 weeks and a 12-week Treatment Period. During the Treatment Period, subjects returned to the clinic weekly for the first 2 visits (Day 8 and Day 15) after the Day 1 visit, followed by visits at Week 4 (Day 29), Week 8 (Day 57), and Week 12 (Day 84) to evaluate safety, tolerability, and preliminary efficacy. Additional assessments included quality of life (QOL) questionnaires, biomarker samples, pharmacokinetics, pharmacodynamics, and optional pharmacogenomics. Safety assessments included physical examinations, vital signs, ECGs, clinical laboratory tests, AEs, and immunogenicity.

Zilucoplan and the matching placebo were supplied as sterile, preservative-free, aqueous solutions prefilled into 1 mL glass syringes with 29 gauge, ½ inch, staked needles placed within self-administration devices. Fill volumes were adjusted based on subject weight range to achieve correct mg/kg dose range. Subjects were instructed to self-administer SC doses daily.

Doses of zilucoplan were determined by target dose and weight, accomplished using fixed dose by weight brackets. These brackets were grouped by body weight category such that each subject received no less than the target minimum dose to avoid sub-therapeutic dosing. For the 0.1 mg/kg dose, subjects received, at a minimum, a fixed dose of 0.1 mg/kg (range: 0.10 to 0.14 mg/kg). For the 0.3 mg/kg dose, subjects received a minimum dose of 0.3 mg/kg (range: 0.30 to 0.42 mg/kg). Table 2 summarizes the dose presentations for zilucoplan 0.1 and 0.3 mg/kg doses. Subjects who presented with a higher body weight (>150 kg) were accommodated on a case-by-case basis. Matching placebo was provided in 2 presentations, 0.220 mL for the 0.1 mg/kg dose and 0.574 mL for the 0.3 mg/kg dose.

TABLE 2
Zilucoplan dose presentations by weight bracket

| | Dose Presentation | | | |
|---|---|---|---|---|
| Target | Fill | | | |
| Dose | Volume | Dose | Weight Range | Dose Range |
| (mg/kg) | (mL) | (mg) | (kg) | (mg/kg) |
| 0.1 | 0.150 | 6 | ≥43 to <61 | 0.10 to 0.14 |
| 0.1 | 0.220 | 8.8 | ≥61 to <88 | 0.10 to 0.14 |
| 0.1 | 0.310 | 12.4 | ≥88 to <109 | 0.11 to 0.14 |
| 0.3 | 0.416 | 16.6 | ≥43 to <56 | 0.30 to 0.39 |
| 0.3 | 0.574 | 23 | ≥56 to <77 | 0.30 to 0.41 |
| 0.3 | 0.810 | 32.4 | ≥77 to ≤150 | 0.30 to 0.42 |

### Screening

Screening was carried out to determine subject study eligibility. Screening included QMG score assessment. The patient population most appropriate for zilucoplan treatment was expected to have a QMG score ≥12 when assessed at screening and baseline (off acetylcholinesterase inhibitor therapy, e.g., pyridostigmine, for at least 10 hours) with ≥4 test items scored at ≥2. Other eligibility criteria assessed during screening included age between 18 and 85; gMG diagnosis [according to Myasthenia Gravis Foundation of America (MGFA) criteria; Class II-IVa] at time of screening; positive serology for AChR autoantibodies; no change in corticosteroid dose for at least 30 days prior to baseline or anticipated to occur during the 12-week Treatment Period; and no change in immunosuppressive therapy, including dose, for at least 30 days prior to baseline or anticipated to occur during the 12-week Treatment Period. Female subjects of childbearing potential needed to have a negative serum pregnancy test at screening and a negative urine pregnancy test within 24 hours prior to the first dose of study drug, sexually active female subjects of childbearing potential (i.e., women who were not postmenopausal or who had not had a hysterectomy, bilateral oophorectomy, or bilateral tubal ligation) and all male subjects (who had not been surgically sterilized by vasectomy) agreed to use effective contraception during the study.

During screening, assessments were performed that included review of medical history and demographics, including collection of disease history with diagnosis of gMG according to MGFA criteria (Class II-IVa): serology for AChR autoantibodies; QMG score assessment; height and weight measurement; assessment of vital signs [heart rate (HR), body temperature, and blood pressure in the sitting position]; 12-lead ECG; assessment of prior Neisseria meningitidis vaccination, collection of blood samples for laboratory testing [hematology, chemistry, coagulation, adenosine deaminase (ADA) testing, and pharmacogenomic analysis]; collection of urine samples for urinalysis; and serum pregnancy testing for females of childbearing potential.

Subjects meeting any of the following criteria were excluded from the study: (1) thymectomy within 6 months prior to baseline or scheduled to occur during the 12-week Treatment Period; (2) abnormal thyroid function as determined by local standard; (3) known positive serology for muscle-specific kinase (MuSK) or lipoprotein receptor-related peptide 4 (LRP4): (4) Minimal Manifestation Status (MMS) of myasthenia gravis based on the clinical evaluation; (5) calculated glomerular filtration rate of <60 mL/min/1.73 m² based on the Modification of Diet in Renal Disease (MDRD) equation at Screening:

$$GFR ( \frac{ml \text{/} min}{1.73 {m}^{2}} ) = 175 \times {( {S}_{cr} )}^{- 1.154} \times {( Age )}^{- 0.203} \times ( 0.742 if female ) \times ( 1.212 if African American ) ; \qquad ( 6 )$$

elevated liver function tests (LFTs) defined as total bilirubin or transaminases [aspartate aminotransferase (AST)/alanine aminotransferase (ALT)]>2 times the upper limit of normal (×ULN); (7) history of meningococcal disease; (8) current or recent systemic infection within 2 weeks prior to baseline or infection requiring IV antibiotics within 4 weeks prior to baseline; (9) pregnant, planning to become pregnant, or nursing female subjects: (10) recent surgery requiring general anesthesia within 2 weeks prior to screening or surgery expected to occur during screening or the 12-week Treatment Period; (11) treatment with an experimental drug or another complement inhibitor within 30 days or 5 half-lives of the experimental drug (whichever is longer) prior to baseline; (12) treatment with rituximab within 6 months prior to baseline; (13) ongoing treatment with IV immunoglobulin G (IVIG) or plasma exchange (PLEX) or treatment within 4 weeks prior to baseline; (14) active neoplasm (other than benign thymoma) requiring surgery, chemotherapy, or radiation within the prior 12 months (subjects with a history of malignancy who have undergone curative resection or otherwise not requiring treatment for at least 12 months prior to screening with no detectable recurrence are allowed); (15) fixed weakness (‘burnt out’ MG) based on clinical judgement; (16) history of any significant medical or psychiatric disorder that render subject unsuitable for participation in the study; and (17) participation in another concurrent clinical trial involving an experimental therapeutic intervention (participation in observational studies and/or registry studies is permitted).

### Treatment Period

Randomized subjects received 0.1 mg/kg zilucoplan, 0.3 mg/kg zilucoplan, or matching placebo administered SC at the Day 1 visit. Following in-clinic education and training, all subjects self-injected daily SC doses of blinded study drug, according to randomized treatment allocation, for the subsequent 12 weeks. An injection device was provided for use during the study. Subjects were expected to remain on stable doses of standard of care (SOC) therapy for gMG throughout the study, including pyridostigmine, corticosteroids, or immunosuppressive drugs. Dosing on study visit days was withheld until QMG scoring and blood collection [for pharmacokinetic (PK) and pharmacodynamic (PD) analysis] was completed. On days when rescue therapy was concurrently administered, study drug dosing was held until after administration of rescue therapy and PK/PD sampling. Rescue therapy involved escalation of gMG therapy due to deterioration of subject clinical status. During rescue therapy, subjects received immunoglobulin (IVIG) or plasma exchange treatment.

During the Main Portion of the study, the total duration of study participation for all subjects was up to approximately 16 weeks, including a Screening Period of up to 4 weeks and a 12-week Treatment Period. A study Extension Portion was made available for continued zilucoplan administration.

Subjects received treatment with 0.1 mg/kg zilucoplan, 0.3 mg/kg zilucoplan, or matching placebo, according to randomization, from Day 1 to Day 84 during the Treatment Period of the Main Portion of the study. Subjects who completed the Day 84 visit (including those randomized to the placebo arm) had the option to continue treatment with zilucoplan in the Extension Portion of the study.

End of Study and Final Study procedures included weight measurement: review and documentation of concomitant medications: symptom-directed physical examination; assessment of vital signs (e.g., heart rate, body temperature, and blood pressure in sitting position); 12-lead ECG; collection of blood samples for laboratory testing (hematology, chemistry, coagulation, ADA testing, pharmacokinetic analysis, pharmacodynamic analysis, and biomarker analysis); collection of urine for urinalysis; urine pregnancy testing for females of childbearing potential; QMG score assessment; and assessment of MG-ADL, MG-QOL15r, and MG composite (MGC).

### Sample Analysis

During the Main Portion of the study, blood samples for PK and PD analysis were collected from all subjects at the time points presented in Table 3. Extension Portion blood sample collection for PK and PD analysis was designed to be identical under rescue therapy scenarios. Additionally, blood taken at Week 36 of the Extension Portion was planned to follow the “Day 1” schedule described for Main Portion blood collection.

TABLE 3
Time points for blood collections

| | | During Rescue Therapy | |
|---|---|---|---|
| | | At sites where rescue | At sites where rescue |
| | | therapy is | therapy is NOT |
| Day 1 | Day 84 | administered locally | administered locally |
| Pre-dose | Pre-dose | Within 1 hour before | Prior to administration |
| (within 1 hour | (any time prior to | administration of | of the first course of |
| before first dose of | Day 84 study drug | rescue therapy | rescue therapy |
| study drug) | administration) | | |
| 1-hour post-dose | 1-hour post-dose | For PK (for plasma | For PK: Prior to |
| (±30 minutes) | (±30 minutes) | exchange only): | administration of the |
| | | Measured in the | first course of rescue |
| | | exchanged plasma | therapy |
| | | For PD: Within 1 hour | For PD: After |
| | | before administration | administration of the |
| | | of rescue therapy | last course of rescue |
| | | | therapy |
| 3 hours post-dose | | Within 1 hour after | After administration |
| (±30 minutes) | | administration of | of the last course of |
| 6 hours post-dose | | rescue therapy | rescue therapy |
| (±90 minutes) | | | |

On all other study visit days, single PK and PD samples were collected prior to administration of study drug. Plasma concentrations of zilucoplan and its metabolites were measured in all blood samples.

Blood samples for safety analyses were collected at the following time points on Day 1: (i) pre-dose (within 1 hour before first dose administration of study drug) and (ii) 6 hours post-dose (±90 minutes). At all other study visits, samples for safety analysis were collected prior to administration of study drug. An additional blood sample for testing was taken at 6 hours post-dose (±90 minutes) on Day 84 from subjects intending to participate in the Extension Portion of the study. Blood sample analytes assessed included those listed in Table 4.

TABLE 4
Blood sample analytes
Analyte
Alanine aminotransferase (ALT)
Albumin
Alkaline phosphatase (ALP)
Amylase
Aspartate aminotransferase (AST)
Bicarbonate
Bile acids
Bilirubin (total, direct, and indirect)
Blood urea nitrogen (BUN)
Calcium
Chloride
Creatinine
Gamma-glutamyl transferase (GGT)
Glucose
Lipase
Potassium
Sodium
Total protein
Uric acid
Hematocrit
Hemoglobin
Mean corpuscular volume (MCV)
Platelet count
White blood cell (WBC) count and differential (%)
International normalized ratio (INR)/prothrombin time (PT)
Fibrinogen
Partial thromboplastin time (PTT) or activated partial
thromboplastin time (aPTT)
C-reactive protein (CRP)
Creatine phosphokinase (CPK)

MG pathophysiology biomarker analysis [e.g., complement fixation, complement function, complement pathway proteins, autoantibody characterization (titer and immunoglobulin class), and inflammatory markers] was available to provide further insight into clinical efficacy and safety of zilucoplan in subjects with gMG. Assessment of complement protein levels and complement activity can be used to evaluate response to zilucoplan and to understand subject characteristics related to variations in drug response. Inflammation marker testing can be used to assess correlation with complement function and clinical response to zilucoplan. A list of analytes can be created through review of the literature, ongoing clinical studies, and ongoing exploratory work and finalized after completion of the study.

The primary efficacy endpoint was the change from baseline to Week 12 (Day 84) in QMG score. The QMG score is a standardized and validated quantitative strength scoring system that was developed specifically for MG and has been used previously in clinical trials. Higher scores are representative of more severe impairment. Recent data suggest that improvements in the QMG score of 2 to 3 points may be considered clinically meaningful, depending upon disease severity [Barohn, R J et al. 1998, Ann N Y Acad Sci. 841:769-72; Katzberg, H D et al. 2014, Muscle Nerve, 49(5):661-5]. QMG assessment was performed at each study visit and at screening to assess subject eligibility. The QMG assessment was performed at approximately the same time of day (preferably in the morning) at each visit throughout the study. If a subject was receiving a cholinesterase inhibitor (e.g., pyridostigmine), the dose was withheld for at least 10 hours prior to QMG test. 0.3 mg/kg and 0.1 mg/kg dose groups were compared to placebo dose group and linear trends were assessed based on all three treatment groups.

Secondary efficacy endpoints included Week 12 change from baseline in MG-ADL, MG-QOL15r, and MG Composite. Each of the active doses was compared to the placebo group. For subjects with ≥3-point reduction in QMG score at Week 12 and subjects requiring rescue therapy over the 12-week Treatment Period, the rate of subjects meeting the endpoint for each of the active treatment groups was compared to the placebo group.

The specific order of efficacy endpoint analyses was arranged to reduce subject fatigue and enhance result reliability. MG-QOL15r analysis was conducted first, followed by MG-ADL analysis, QMG score assessment, and MG Composite. The same evaluator was used throughout the study to reduce assessment variability.

### Results

Study results from a broad, demographically well balanced population of participants were obtained. Pre-study baseline characteristics for study participants are presented in Table 5. In the Table, “SD” refers to “standard deviation” and “SOC” refers to “standard of care.”

TABLE 5
Baseline study participant characteristics

| | | | 0.1 mg/kg | | 3 mg/kg | |
|---|---|---|---|---|---|---|
| | Placebo | | zilucoplan | | zilucoplan | |
| Characteristic | (n = 15) | | (n = 15) | | (n = 14) | |
| Age, years, mean (SD) | 48.4 | (15.7) | 45.5 | (15.7) | 54.6 | (15.5) |
| Male, n (%) | 4 | (26.7%) | 7 | (46.7%) | 10 | (71.4%) |
| Weight, kg, mean (SD) | 85.27 | (21.44) | 93.71 | (24.72) | 110.9 | (30.79) |
| BMI, mean (SD) | 30.856 | (7.386) | 32.804 | (6.548) | 36.000 | (8.242) |
| Race, n (%) | | | | | | |
| White | 12 | (80.0%) | 13 | (86.7%) | 11 | (78.6%) |
| Asian | 1 | (67%) | 0 | | 1 | (7.1%) |
| Black or African American | 2 | (13.3%) | 2 | (13.3%) | 2 | (14.3%) |
| MGFA Class at Screening | | | | | | |
| II | 7 | (46.7%) | 5 | (33.3%) | 5 | (35.7%) |
| III | 8 | (53.3%) | 10 | (66.7%) | 5 | (35.7%) |
| IV | 0 | | 0 | | 4 | (28.6%) |
| Age at Disease Onset, years, mean (SD) | 40.3 | (17.79) | 37.3 | (16.04) | 46.9 | (19.48) |
| Duration of Disease, years, mean (SD) | 8.04 | (0.1, 20.9) | 8.71 | (1.6, 24.1) | 8.3 | (0.5, 26,0) |
| Baseline QMG Score, mean (SD) | 18.7 | (4.0) | 18.7 | (4.0) | 19.1 | (5.1) |
| Baseline MG-ADI, Score, mean (SD) | 8.8 | (3.6) | 6.9 | (3.3) | 7.6 | (2.6) |
| Baseline MGC Score, mean (SD) | 18.7 | (5.7) | 14.5 | (6.3) | 14.6 | (6.3) |
| Baseline MG-QOL15r Score, mean (SD) | 15.9 | (7.4) | 19.1 | (5.0) | 16.5 | (7.3) |
| Prior MG Therapies (Standard of Care) | | | | | | |
| Pyridostigmine, n (%) | 14 | (93.3%) | 15 | (100%) | 14 | (100%) |
| Corticosteroids, n (%) | 13 | (86.7%) | 13 | (86.7%) | 14 | (100%) |
| Immunosuppressants, n (%) | 12 | (80.0%) | 12 | (80.0%) | 9 | (64.3%) |
| Prior IVIG, n (%) | 9 | (60.0%) | 8 | (53.3%) | 10 | (71.4%) |
| Prior Plasma Exchange, n (%) | 7 | (46.7%) | 9 | (60.0%) | 7 | (50.0%) |
| Prior Thymectomy, n(%) | 5 | (33.3%) | 8 | (53.3%) | 7 | (50.0%) |
| Prior MG crisis requiring intubation, n (%) | 3 | (20.0%) | 4 | (26.7%) | 2 | (14.3%) |

The population included subjects with baseline disease characteristics indicative of refractory as well as non-refractory disease status. Baseline disease characteristics including MGFA classification and efficacy outcome measures were also well balanced among study participants. In the study, 15 subjects received placebo, while 15 subjects received low dose zilucoplan (0.1 mg/kg) and 14 subjects received high dose zilucoplan (3 mg/kg). Significance testing was pre-specified at a 1-sided alpha of 0.1.

Baseline demographic characteristics were similar across groups with respect to mean age (48.4 to 54.6 years years), race representation (78.6% to 86.7% white), mean weight (85.27 to 110.9 kg) and mean BMI (30.856 to 36.000). There was an imbalance between the groups with respect to gender 71.4%, 46.7%, and 26.7% male in the 0.3 mg/kg zilucoplan, 0.1 mg/kg zilucoplan, and placebo groups, respectively. However, gender is not known to play a significant role in treatment response in gMG.

Medical history including duration of disease, prior MG crisis, prior thymectomy, and prior treatment with pyridostigmine, corticosteroid therapy, immunosuppressive agents, or rescue therapy with WIG or PLEX was well balanced across treatment groups. Over 90% of subjects in each group had received acetylcholinesterase inhibitors; over 85% had received corticosteroids; 64.3 to 80% had received immunosuppressive therapy; 53.3 to 71.4% had received IVIG; and 46.7 to 60.0% had undergone plasma exchange.

MG disease severity as measured by MGFA classification was similar across the treatment groups with all subjects in the 0.1 mg/kg zilucoplan and placebo groups being in MGFA class 11 (mild disease severity) and III (moderate disease severity), although the 0.3 mg/kg zilucoplan group also included four subjects in MGFA class IV (severe disease). MG specific baseline characteristics were well balanced across the primary (QMG) and first secondary (MG-ADL) endpoint scores, with mean baseline QMG scores of 19.1, 18.7, and 18.7; and mean MG-ADL scores of 7.6, 6.9, and 8.8 in the 0.3 mg/kg zilucoplan, 0.1 mg/kg zilucoplan, and placebo groups, respectively. The MG-QOL15r was approximately three points higher in the 0.1 mg/kg zilucoplan group than in the 0.3 mg/kg zilucoplan group with mean MG-QOL15r scores of 16.5, 19.1, and 15.9 in the 0.3 mg/kg zilucoplan, 0.1 mg/kg zilucoplan, and placebo groups, respectively. The MGC was >4 points higher in the placebo group than in the other two groups with mean MGC scores of 14.6, 14.5, and 18.7 in the 0.3 mg/kg zilucoplan, 0.1 mg/kg zilucoplan, and placebo groups, respectively.

Clinical efficacy outcomes are provided in Table 6. In the Table, P-values are one-sided based on analysis of covariance (ANCOVA) model, with baseline values as covariates and using last observation carried forward (LOCF) for subjects who received rescue therapy. “LS” refers to “least squares,” “CFB” refers to change from baseline, and “se” refers to “standard error.”

TABLE 6
Clinical efficacy outcomes

| Value | QMG | MG-ADL | MG-QOL15r | MGC |
|---|---|---|---|---|
| 0.3 mg/kg zilucoplan CFB LS mean (se) | −6.0 (1.2) | −3.4 (0.9) | −5.9 (1.7) | −7.4 (1.6) |
| 0.1 mg/kg zilucoplan CFB LS mean (se) | −5.5 (1.2) | −3.3 (0.9) | −7.4 (1.7) | −5.3 (1.5) |
| Placebo LS mean CFB (se) | −3.2 (1.2) | −1.1 (0.9) | −2.1 (1.7) | −3.3 (1.6) |
| 0.3 mg/kg zilucoplan CFB LS mean | −2.8 (1.7) | −2.3 (1.3) | −3.7 (2.4) | −4.1 (2.2) |
| difference vs. placebo | | | | |
| p-value | 0.05 | 0.04 | 0.06 | 0.04 |
| 0.1 mg/kg zilucoplan CFB LS mean | −2.3 (1.7) | −2.2 (1.3) | −5.3 (2.4) | −2.0 (2.2) |
| difference vs. placebo (se) | | | | |
| p-value | 0.09 | 0.05 | 0.02 | 0.19 |

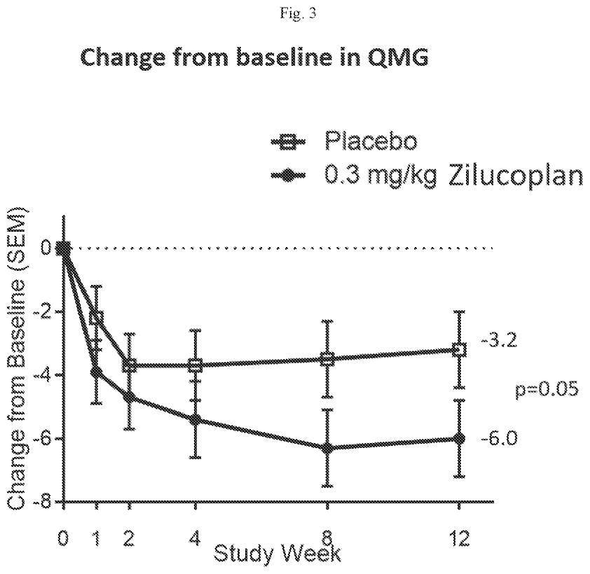

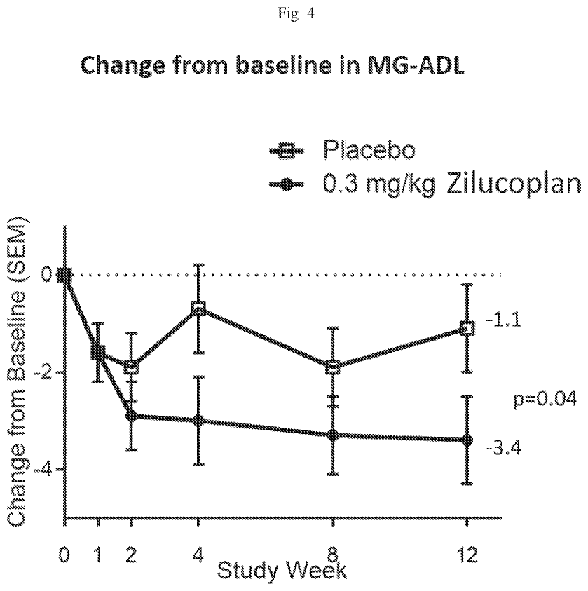

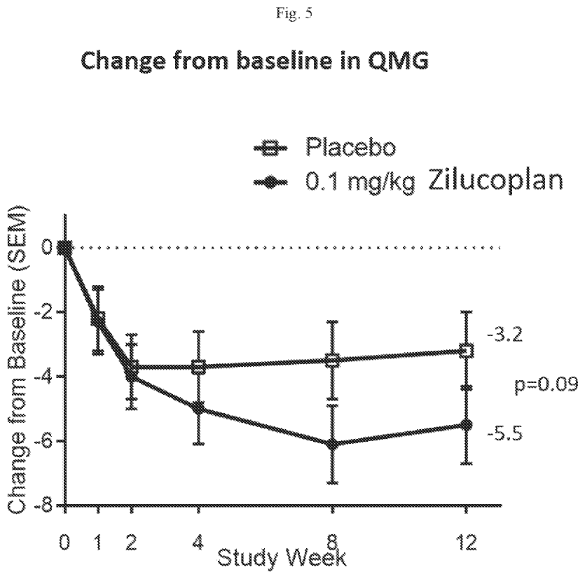

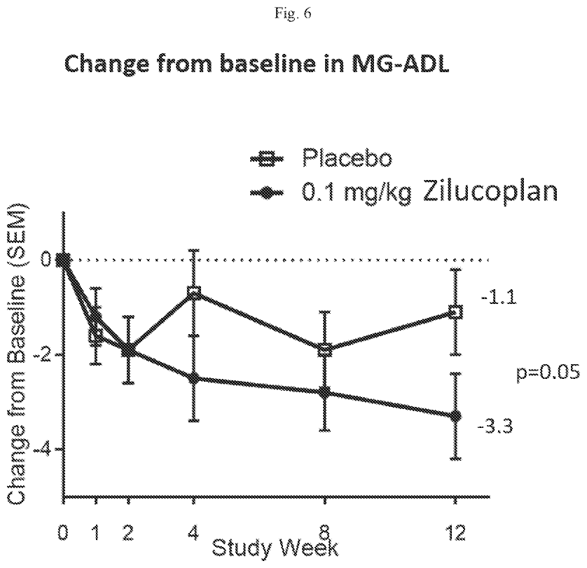

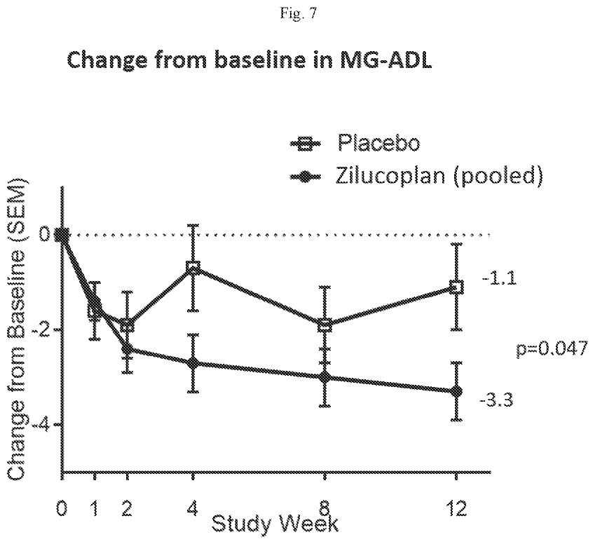

0.3 mg/kg treatment groups showed clinically meaningful and statistically significant improvement in QMG score (≥3 points) over baseline at the 12-week time point (FIG. 3), with mean difference over placebo of −2.8. Clinically meaningful and statistically significant improvement in MG-ADL score (≥2 points) over baseline was also observed with this treatment group at week 12 (FIG. 4), with mean difference over placebo of −2.3. Clinically meaningful and statistically significant improvements were also observed in the low dose treatment group, demonstrating only slightly lower changes from baseline than those observed with higher dose subjects. With lower dose treatment (0.1 mg/kg), clinically meaningful and statistically significant improvement in QMG score was observed with −2.3 point mean difference over placebo at week 12 (FIG. 5). Clinically meaningful and statistically significant changes in MG-ADL score at week 12 were also observed with this group (−2.2 mean difference over placebo; FIG. 6). Changes in MG-ADL from baseline for placebo versus mean changes of the pooled low and high dose zilucoplan treatment groups (n=29) are shown in FIG. 7 and show statistically significant advantage for zilucoplan treatment over placebo (−2.2 mean difference over placebo; p=0.047, 2-sided). In comparing treatment responders to placebo responders in the high dose group to placebo at 12 weeks, the highest levels of improvements in QMG score and MG-ADL were all in the zilucoplan treatment group and there were generally more patients on zilucoplan improving at each cut-off level compared to placebo.

Zilucoplan reduced the need for rescue treatment with only one subject (7%) in the low dose treatment group and zero subjects in the high dose treatment group requiring rescue (as compared to three subjects (20%) requiring rescue therapy in the placebo group). No significant endpoint differences were observed between treatment groups based on prior therapy covariates (immunosuppressive therapy, IVIG, or PLEX), all with P values above 0.20.

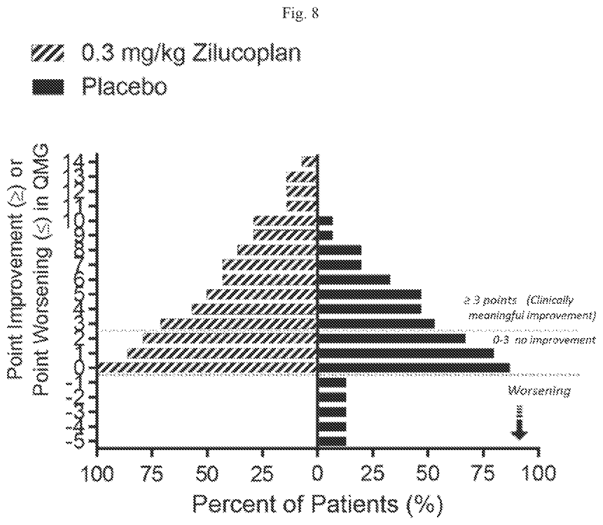

Responder analysis was conducted for QMG and MG-ADL endpoints. A clinically meaningful response on the QMG total score was defined as an improvement of three points or more, in line with the higher end of the established minimal clinically important difference (MCID) for QMG (Barohn et al. 1998; Katzberg et al. 2014). The proportion of responders at week 12 using the cut-off of ≥3 points on QMG was higher for subjects receiving 0.3 mg/kg zilucoplan (n=10/14) and 0.1 mg/kg zilucoplan (n=10/15) compared to the placebo (n=8/15) group (FIG. 8). Additional pre-planned analyses showed an advantage for the zilucoplan treated groups at all cut-offs on QMG, including no subject with worsening in the 0.3 mg/kg zilucoplan group compared to three and two subjects in the 0.1 mg/kg zilucoplan and placebo groups, respectively. None of these differences were statistically significant except for the 0.1 mg/kg zilucoplan group vs. placebo at the ≥7 points and ≥11 points improvement cut-offs (Table 7), no correction for multiple testing was performed. Overall, the data were consistent with the primary analysis, generally showing higher clinical response in the zilucoplan treated arms than in the placebo group.

TABLE 7
Proportion of subjects with improved/worsened QMG
scores at week 12

| | 0.3 mg/kg | 0.1 mg/kg | |
|---|---|---|---|
| | zilucoplan | zilucoplan | Placebo |
| Cut-off | n = 14 | n = 15 | n = 15 |
| Worsened | 0 (0%) | 3 (20.0%) | 2 (13.3%) |
| ≥3 points improvement | 10 (71.4%) | 10 (66.7%) | 8 (53.3%) |
| ≥7 points improvement | 6 (42.9%) | 8 (53.3%)* | 3 (20.0%) |
| ≥11 points improvement | 2 (14.3%) | 4 (26.7%)* | 0 (0%) |

*p < 0.1 (Fischer's exact test, one-sided p-value comparison of each zilucoplan dose group versus placebo).

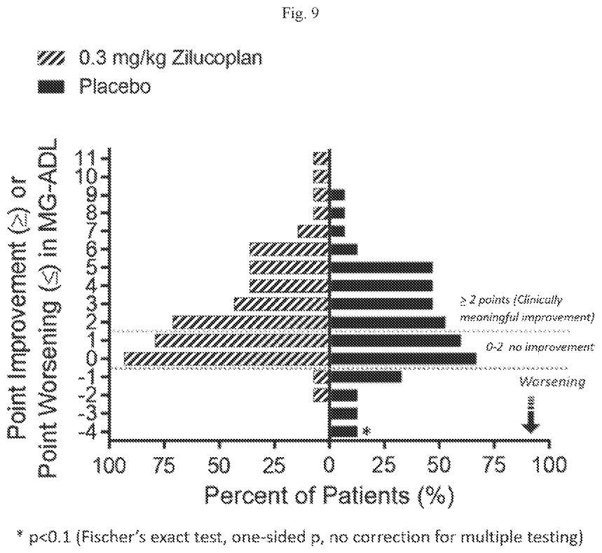

The generally accepted MCID on the MG-ADL total score is an improvement of two points or more (Wolfe et al. 1999: Muppidi et al. 2011). Analyses also included higher cut-offs up to a difference of at least 11 points. The proportion of responders at week 12 using the cut-off of ≥2 points on MG-ADL was higher for subjects receiving zilucoplan at 0.3 mg/kg (n=10/14, 71.4%) and 0.1 mg/kg (n=10/15) compared to the placebo (n=8/15) group (FIG. 9 and Table 8).

TABLE 8
Proportion of subjects with improved/worsened MG-ADL
scores at week 12

| | 0.3 mg/kg | 0.1 mg/kg | |
|---|---|---|---|
| | zilucoplan | zilucoplan | Placebo |
| Cut-off | n = 14 | n = 15 | n = 15 |
| Worsened | 1 (7.1)%* | 1 (6.7%)* | 5 (33.3%) |
| ≥2 points improvement | 10 (71.4%) | 9 (60.0%) | 8 (53.3%) |

*p < 0.1 (Fischer's exact test, one-sided p, no correction for multiple testing).

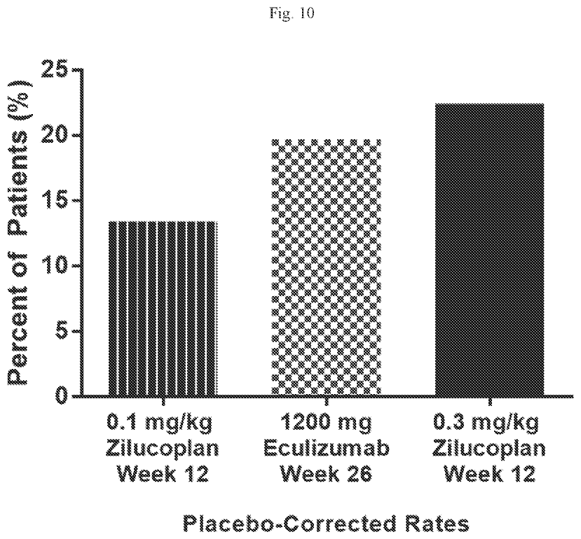

A minimal symptom expression (MSE) endpoint was assessed to determine how many subjects become free or virtually free of MG symptoms (based on achieving an MG-ADL total score of 0 or 1) with zilucoplan therapy. In this study 35.7% (5/14) subjects in the 0.3 mg/kg zilucoplan group achieved an MG-ADL of 0 or 1, compared to 26.7% (4/15) in the 0.1 mg/kg zilucoplan and 13.3%(2/15) in the placebo group (placebo-corrected rates shown in FIG. 10). Further, the percent achievement for the high dose treatment group was greater than that observed with 26 weeks eculizumab treatment (based on eculizumab study results presented in Vissing, J. et al., 2018. AANEM Abstract 193). This analysis underscored the large extent to which improvement of subjective perception of disease burden can be achieved within a short period of time with zilucoplan. The dose response with a higher proportion of patients achieving MSE in the 0.3 mg/kg zilucoplan group was again evident in this analysis.

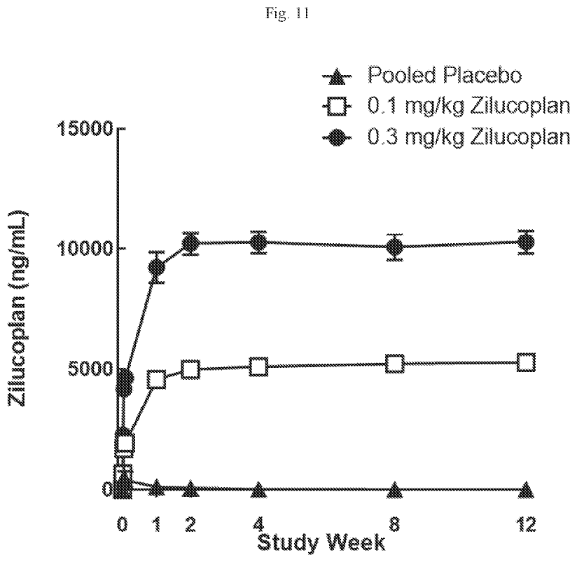

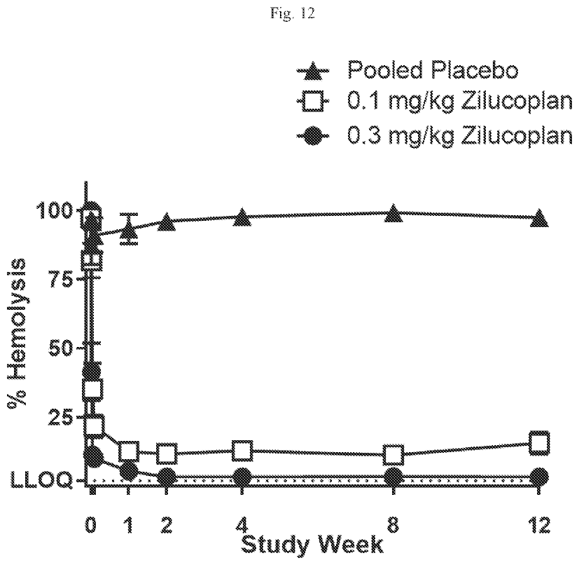

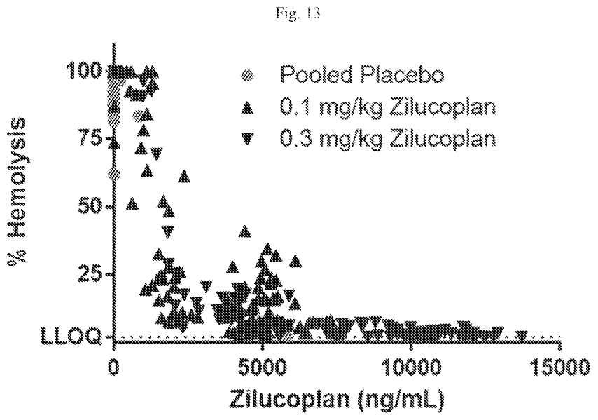

Sparse sampling was performed for assessment of pharmacokinetic and pharmacodynamic data. Steady state zilucoplan plasma concentrations were achieved within the first two weeks of treatment, and no further accumulation was observed (FIG. 11). Trough levels of zilucoplan at steady state (at two weeks or later) in the 0.3 mg/kg zilucoplan dose group ranged between 7,168 ng/mL and 13,710 ng/mL while they were between 2,364 ng/mL and 7,290 ng/mL in the 0.1 mg/kg zilucoplan dose group. 0.3 mg/kg zilucoplan dose consistently achieved complete terminal complement pathway inhibition as measured by sheep red blood cell (sRBC) hemolysis assay (≥95% inhibition at trough). The 0.1 mg/kg dose of zilucoplan, by contrast, did not consistently achieve complete hemolysis inhibition (FIG. 12). Pharmacokinetic and pharmacodynamic results were closely correlated in patients with gMG (FIG. 13), indicating complete inhibition of the terminal complement pathway with zilucoplan plasma concentrations above approximately 9,000 ng/ml.

Overall, both high and low dose zilucoplan treatments were effective, with minimal adverse effects and higher doses yielding more robust clinical improvement.

### Example 4. Extension Portion

At the conclusion of the Treatment Period in the Main Portion of the study described in the above Example, all subjects were given the option to receive zilucoplan in an Extension Portion of the study provided they met Extension Portion selection criteria. Subjects assigned to a zilucoplan treatment arm during the Main Portion of the study continued to receive the same dose of study drug during the Extension Portion. Subjects assigned to the placebo arm during the Main Portion of the study were randomized in a 1:1 ratio to receive daily SC doses of 0.1 mg/kg zilucoplan or 0.3 mg/kg zilucoplan. Assessments and visits during the first 12 weeks of the Extension Portion were identical to the Main Portion of the study for all subjects to ensure appropriate monitoring of subjects transitioning from placebo to active treatment and to maintain blinding of treatment assignment.

Selection criteria for inclusion in the Extension Portion included: (1) positive serology for AChR autoantibodies; (2) negative serum pregnancy test at screening for female subjects of childbearing potential and a negative urine pregnancy test within 24 hours prior to the first dose of study drug; (3) agreement to use effective contraception during the study for sexually active female subjects of childbearing potential (i.e., women who are not postmenopausal or who have not had a hysterectomy, bilateral oophorectomy, or bilateral tubal ligation) and all male subjects (who have not been surgically sterilized by vasectomy); (4) use of any disallowed medications per the exclusion criteria from the Main Portion of the study or altered dosing of any other concomitant medication, unless medically indicated; (5) and no new medical conditions since entry into the Main Portion of the study.

During the Extension Portion of the study, biopsy sections obtained from subjects undergoing thymectomy, lymphadenectomy, or other surgical excision were sent for exploratory immunohistochemical and biomarker analysis.

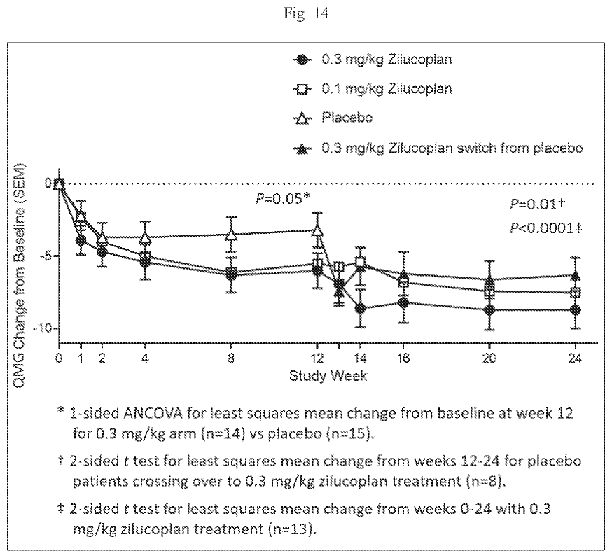

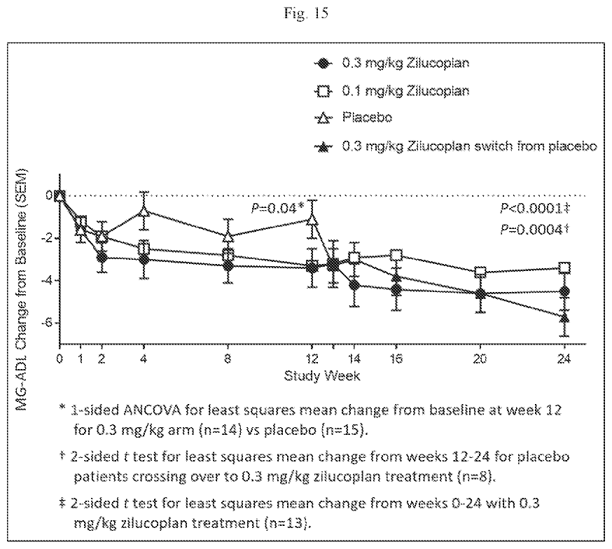

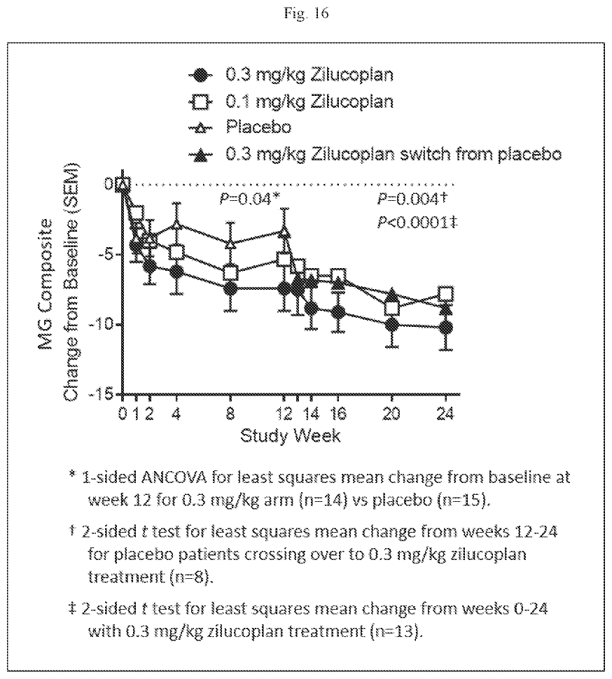

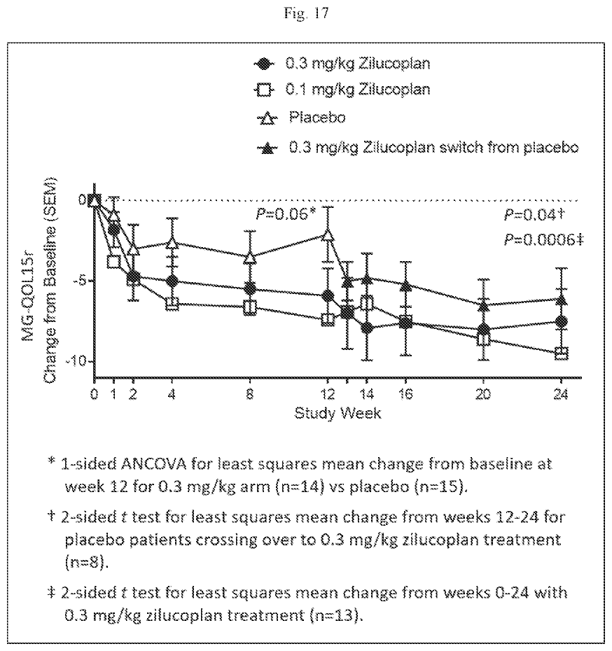

41 patients completed the 12-week Extension Portion of the study (24 weeks total treatment period). Sustained responses were observed for each of: (1) QMG, 8.7 point reduction over baseline, p<0.0001 (FIG. 14); (2) MG-ADL, 4.5 point reduction over baseline, p<0.0001 (FIG. 15): (3) MG Composite, 10.2 point reduction over baseline, p<0.0001 (FIG. 16); and MG-QOL 15r, 7.5 point reduction over baseline, p=0.0006 (FIG. 17).

Placebo subjects crossing over to active drug after 12 weeks also experienced rapid, clinically meaningful, and statistically significant improvement for each endpoint: (1) QMG, 3.1 point reduction over pretreatment level (level associated with 12 week placebo treatment), p=0.01 (FIG. 14); (2) MG-ADL, 3.6 point reduction over pretreatment level, p=0.0004 (FIG. 15); (3) MG Composite, 5.5 point reduction over pretreatment level, p=0.004 (FIG. 16); and MG-QOL 15r, 4.0 point reduction over pretreatment level, p=0.04 (FIG. 17).

### Example 5. Pharmacogenomic Analysis

Pharmacogenomic analysis is carried out on blood samples obtained at screening. Genomic studies [e.g., deoxyribonucleic acid (DNA) sequencing, DNA copy number analysis, and ribonucleic acid expression profiling] are performed and include exploration of whether specific genomic features correlate with response or resistance to study drug.

### Example 6. Urinalysis

Urinalysis is performed on samples collected during screening and during all study and rescue therapy visits to assess pH, specific gravity, protein (qualitative), glucose (qualitative), ketones (qualitative), bilirubin (qualitative), urobilinogen, occult blood, hemoglobin, and cells. A microscopic examination is performed where necessary to confirm measurement.

### Example 7. Phase III Study

A single, global, randomized, double-blind, placebo-controlled, parallel-group, multicenter trial (n=130) testing 0.3 mg/kg zilucoplan daily subcutaneous treatment versus placebo over 12-weeks is conducted to evaluate efficacy of zilucoplan in subjects with gMG. After the 12-week double-blind, placebo-controlled period, all subjects have the option of receiving zilucoplan in an open label study extension. Subjects are evaluated for changes in MG-ADL (primary endpoint), QMG, MG Composite, and MG-QOL15r during and after main study and extension study treatments.

Patients with gMG are enrolled in the study, subject to satisfying the main inclusion criteria: (1) MGFA clinical classification Class II to IV; (2) positive serologic test for anti-AChR antibodies; (3) MG-ADL score ≥5; (4) QMG total score ≥12: (5) no change in corticosteroid dose or immunosuppressive therapy for at least four weeks prior to randomization or anticipated to occur during the 12-week Treatment Period; and have not undergone PLEX or received IVIG for at least four weeks and rituximab for at least 12 months prior to dosing. Selection is based on anti-AChR positive status to ensure that patients who are expected not to respond to complement inhibitor therapy due to absence of complement binding antibodies, such as anti-MUSK-antibody positive patients, are excluded; and to ensure that all patients who enter the study have clinically as well as laboratory confirmed diagnosis of MG. The study population is not restricted to ‘treatment refractory’ patients and enrollment of patients across the disease spectrum is allowed. There is no mechanistic basis to believe that terminal complement inhibition is effective only in patients who have exhausted all other therapies.

### Example 8. Zilucoplan Inhibits Autoantibody-Induced Complement Activity at the Neuromuscular Junction (NMJ)

Co-cultures of human myotubes and neuroblastoma cells were prepared and cultured with human sera as an in vitro NMJ model. Cells were cultured with or without 10 μM zilucoplan and anti-acetyl choline receptor (AChR) 637 antibodies of either IgG1 or IgG4 format. The IgG1 antibodies are known to facilitate complement-mediated C5b-9 deposition, while the IgG4 antibodies do not. Subsequent deposition of C5b-9 was observed by immunofluorescence using an anti-C5b-9 antibody (aE11, AbCam, Cambridge, UK). C5b-9 deposition was observed in cells cultured with anti-AChR 637 IgG1, but without zilucoplan. C5b-9 deposition was absent in cells cultured under the same conditions, but with 10 μM zilucoplan. As complement-mediated destruction of the NMJ contributes to the pathogenesis of gMG, this data exemplifies a mechanistic rationale for positive clinical responses observed in human studies described above.

### Example 9. Zilucoplan Permeability

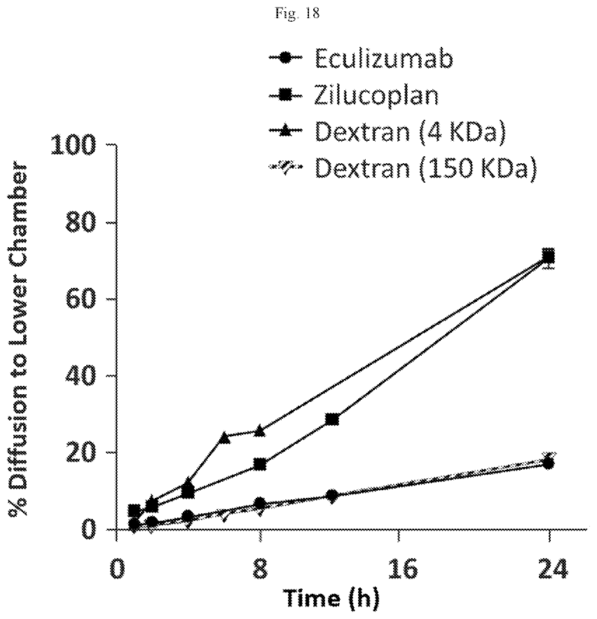

Zilucoplan in-vitro permeability was assessed using a basement membrane model. In the model, diffusion of zilucoplan across an extracellular matrix (ECM) gel membrane (prepared as described in Arends, F. et al. 2016. IntechOpen, DOI: 10.5772/62519) was assessed and compared with diffusion of eculizumab. In the model, compounds were introduced to an upper reservoir, which was separated from a lower reservoir by the ECM gel membrane. The ECM gel membrane was prepared to include matrix components mimicking those found in the basal lamina of neuromuscular junctions. Permeability of the compounds across the membrane was assessed by detection in the lower reservoir. Greater than 20% of the zilucoplan introduced to the upper reservoir had diffused to the lower reservoir after 12 hours and more than 60% by 24 hours (see FIG. 18). In contrast, less than 20% of eculizumab diffused to the lower reservoir after 24 hours. The results demonstrate superior permeability of zilucoplan across the basement membrane as compared with eculizumab (about four times higher), suggesting preferential tissue penetration.

Enhanced permeability of zilucoplan was confirmed by quantitative whole body analysis (QWBA). For this study, zilucoplan C-terminal lysine was radiolabeled with ¹⁴C and administered to rats. Animals were imaged to determine concentration of radiolabeled zilucoplan over time (24 hours) in multiple organs and tissues. Area under the concentration curve (AUC) for each organ or tissue analyzed was expressed as a percentage of plasma AUC to yield a biodistribution value, presented in Table 9 below. Inferred eculizumab biodistribution values, based on monoclonal antibody biodistribution studies published in Shah, D. K., et al. 2013. mAbs. 5:297-305, are provided for comparison.

TABLE 9
Biodistribution comparison

| | Eculizumab | Zilucoplan |
|---|---|---|
| | biodistribution | biodistribution |
| Organ/tissue | % | % |
| Lung | 14.9 | 37.5 |
| Heart | 10.2 | 22.9 |
| Muscle | 3.97 | 7.0 |
| Small intestine | 5.22 | 10.9 |
| Large Intestine | 5.03 | 21.7 |
| Spleen | 12.8 | 15.5 |
| Liver | 12.1 | 27.1 |
| Bone | 7.27 | 15.3 |
| Stomach | 4.98 | 8.5 |
| Lymph nodes | 8.46 | 12.8 |
| Fat | 4.78 | 16.2 |
| Brain | 0.35 | 0.9 |
| Pancreas | 6.4 | 15.8 |
| Testes | 5.88 | 15.5 |
| Thymus | 6.62 | 7.8 |

These results support the use of zilucoplan to inhibit C5 activity in tissues where C5 inhibitor tissue-penetration is needed and wherein eculizumab tissue-penetration is insufficient.

### Example 10. Zilucoplan Drug-Drug Interactions

Zilucoplan in vivo drug-drug interaction studies were carried out with potential comedications in non-human primates. The first investigated the effects of cyclosporine A on the pharmacokinetics of zilucoplan and vice versa. Cyclosporine A is a known inhibitor of organic anion transporting polypeptide (OATP) 1B1 and OATP1B3 and is a potential comedication in PNH. No effects on zilucoplan exposure were observed following cyclosporine A administration, and no effects on cyclosporin A exposure were observed following zilucoplan administration. These results support methods of treating complement-related indications (e.g., myasthenia gravis) in subjects by combined administration of zilucoplan and cyclosporin A.

The second in vivo drug-drug interaction study was performed with zilucoplan and an inhibitor of neonatal Fc receptor (FcRN) recycling, DX-2507, a functionally equivalent variant of DX-2504 with Cys to Ala mutations to improve manufacturing (described in Nixon, A. E. et al. 2015. Front Immunol. 6:176). By inhibiting FcRN, DX-2504 inhibits Fc-mediated recycling, thereby reducing the half-life of IgG antibodies. Administration of DX-2504 serves as a model for intravenous immunoglobulin (IVIG) treatment, which reduces the half-life of IgG antibodies by overwhelming the Fc recycling mechanism with large doses of immunoglobulin. Zilucoplan pharmacokinetics and pharmacodynamics did not change with concomitant dosing of anti-FcRN monoclonal antibody DX-2507 in Cynomolgus monkeys. In addition, no changes in zilucoplan levels were observed in a patient receiving concomitant therapeutic doses of IVIG. These results indicate no effects of FcRN inhibition on zilucoplan pharmacokinetics and support methods of treating complement-related indications (e.g., myasthenia gravis) in subjects by combined administration of zilucoplan and FcRN inhibitor (DX-2504. DX-2507, or IVIG).

1. A method of treating myasthenia gravis (MG), the method comprising administering zilucoplan to a subject.

2. The method of claim 1, wherein the MG is generalized MG (gMG).

3. The method of claim 1, wherein zilucoplan administration comprises subcutaneous (SC) administration.

4. The method of claim 1, wherein zilucoplan is administered at a dose of from about 0.1 mg/kg (mg zilucoplan/kg subject body weight) to about 0.6 mg/kg.

5. (canceled)

6. The method of claim 1, wherein zilucoplan administration comprises use of a prefilled syringe.

7-11. (canceled)

12. The method of claim 6, wherein the prefilled syringe comprises an aqueous solution, the aqueous solution comprising from about 4 mg/ml to about 200 mg/ml zilucoplan in phosphate-buffered saline (PBS), wherein the aqueous solution is optionally preservative free.

13. (canceled)

14. (canceled)

15. The method of claim 12, wherein the aqueous zilucoplan solution comprises a volume of from about 0.15 ml to about 0.81 ml.

16. The method of claim 1, wherein the subject is screened prior to zilucoplan administration, the screening comprising one or more of:
assessment of subject Quantitative Myasthenia Gravis (QMG) score;
selection based on subject QMG score ≥12;
selection based on subject age between 18 and 85 years old;
selection based on subject prior gMG diagnosis, wherein the gMG diagnosis is optionally made according to Myasthenia Gravis Foundation of America (MGFA) criteria;
assessment of subject acetylcholinesterase receptor (AChR) autoantibody levels;
selection based on no change in corticosteroid dose and/or immunosuppressive therapy for at least 30 days prior to screening; and
serum pregnancy testing and/or urine pregnancy testing.

17. (canceled)

18. (canceled)

19. The method of claim 16, wherein the screening comprises assessment of subject QMG score and selection based on subject QMG score of ≥12, wherein:
the subject does not receive MG therapy for at least 10 hours prior to QMG score assessment;
the subject does not receive acetylcholinesterase inhibitor therapy for at least 10 hours prior to QMG score assessment; and/or
≥4 QMG test items achieve a score of ≥2.

20-28. (canceled)

29. The method of claim 1, wherein zilucoplan administration comprises daily administration.

30. The method of claim 2, wherein the subject simultaneously receives standard of care gMG therapy.

31. The method of claim 30, wherein the standard of care gMG therapy comprises one or more of pyridostigmine treatment, corticosteroid treatment, and immunosuppressive drug treatment.

32. The method of claim 1 the method comprising evaluating or monitoring the subject for an MG characteristic during or after zilucoplan treatment, wherein the MG characteristic comprises one or more of QMG score, Myasthenia Gravis-Activities of Daily Living (MG-ADL) score, MG-QOL15r score, and MG Composite score.

33. (canceled)

34. The method of claim 32, wherein subject QMG score is reduced during or after zilucoplan treatment.

35. The method of claim 34, wherein subject QMG score is reduced by at least 3 points at or before 12 weeks of zilucoplan treatment.

36. (canceled)

37. The method of claim 32, wherein the subject is receiving cholinesterase inhibitor treatment over the course of zilucoplan treatment and wherein the cholinesterase inhibitor treatment is withheld for at least 10 hours prior to evaluating or monitoring the subject for the MG characteristic.

38. (canceled)

39. (canceled)

40. The method of claim 32, wherein the MG characteristic comprises:
change in MG Composite score of at least 3 points from a baseline MG Composite score at or before 12 weeks of zilucoplan treatment; and/or
change in MG-ADL score of at least 2 points from a baseline MG-ADL score at or before 12 weeks of zilucoplan treatment.

41-45. (canceled)

46. The method of claim 32, wherein the zilucoplan is administered at a daily dose of from about 0.1 mg/kg to about 0.6 mg/kg by subcutaneous injection of a zilucoplan solution, the zilucoplan solution comprising about 40 mg/ml zilucoplan in PBS.

47. The method of claim 1, wherein subject zilucoplan plasma levels reach maximum concentration (Cₘₐₓ) on the first day of treatment.

48. The method of claim 1, wherein at least 90% hemolysis inhibition is achieved in subject serum, wherein, optionally, hemolysis inhibition is measured by a sheep red blood cell (sRBC) hemolysis assay.

49-60. (canceled)

61. A method of evaluating a treatment for MG, the method comprising:
screening an evaluation candidate for at least one evaluation participation criteria;
selecting an evaluation participant;
administering the treatment for MG to the evaluation participant; and
assessing at least one efficacy endpoint;
wherein, optionally, the treatment for MG comprises administering zilucoplan.

62-112. (canceled)

113. An administration device prepared for treatment of MG, the administration device comprising:
a self-injection device comprising a syringe and needle; and
a predetermined volume of a pharmaceutical composition, wherein the pharmaceutical composition comprises a 40 mg/mL concentration of zilucoplan in an aqueous solution, and wherein the predetermined volume is modified to facilitate zilucoplan administration to a subject at a dose of 0.3 mg per kg subject weight.

114. (canceled)

115. A kit prepared for treatment of MG, the kit comprising:
a set of administration devices, wherein each member of the set of administration devices comprises a self-injection device, the self-injection device comprising a syringe, a needle, and a predetermined volume of a zilucoplan solution, wherein the predetermined volume of the zilucoplan solution is modified to facilitate zilucoplan administration to a subject at a dose of 0.3 mg per kg subject weight, and wherein the zilucoplan solution comprises 40 mg/mL zilucoplan in PBS; and
instructions for use of the kit.

116-120. (canceled)


SEQUENCE LISTING (1 sequence)

SEQ ID NO: 1 - protein, 15 aa, Artificial Sequence
  source: /note=“Description of Artificial Sequence Synthetic peptide”
  N-term Ac
  SITE (1)..(6): /note=“Lactam bridge between residues”
  MOD_RES (7)..(7): N-methyl-aspartic acid
  MOD_RES (8)..(8): Tert-butylglycine
  MOD_RES (10)..(10): 7-azatryptophan
  MOD_RES (14)..(14): Cyclohexylglycine
  MOD_RES (15)..(15): N-epsilon-(PEG24-gamma-glutamic acid-N-alpha-hexadecanoyl)Lys
KVERFDXXYXEYPXK